(19) United States
(12) Patent Application Publication
(10) Pub. No.: US 2015/0320592 A1
(43) Pub. Date: Nov. 12, 2015

(54) Devices, Systems and Methods for Delivering Thermal Stimulation

(71) Applicant: Scion NeuroStim, LLC, Raleigh, NC (US)

(72) Inventors: Robert D. Black, Chapel Hill, NC (US); Lesco L. Rogers, Raleigh, NC (US); Lanty L. Smith, Raleigh, NC (US)

(21) Appl. No.: 14/707,428

(22) Filed: May 8, 2015

Related U.S. Application Data
(60) Provisional application No. 61/990,794, filed on May 9, 2014.

(51) Int. Cl. A61F 7/12

(52) U.S. Cl. CPC A61F 7/12; A61F 2007/0005

(57) ABSTRACT

The present provides devices, systems and methods useful for stimulating the vestibular and/or nervous systems of an individual with a gas or mixture of gases that has been cooled, heated and/or humidified to produce one or more time-varying thermal waveforms.

### RELATED APPLICATION

This application claims priority from U.S. Provisional Application No. 61/990,794, filed May 9, 2014, the disclosure of which is hereby incorporated herein in its entirety.

### FIELD OF THE INVENTION

The present invention relates to devices, systems and methods for stimulating the vestibular and/or nervous systems of an individual.

### BACKGROUND

Caloric vestibular stimulation (“CVS”) has long been known as a diagnostic procedure for testing the function of the vestibular system. In the traditional hospital setting, water caloric tests are used to assess levels of consciousness following acute or chronic brain injury. The brain injury may be due to head trauma or a central nervous system event such as a stroke. Other brain injuries occur in the presence of metabolic abnormalities (e.g., kidney disease, diabetes), seizures, or toxic levels of controlled substances or alcohol. CVS may also be used to evaluate the integrity of the vestibular organs. Patients with balance difficulties are often evaluated with CVS. Conventional diagnostic CVS systems typically deliver water or a gas at a constant temperature to the ear.

U.S. Patent Publication No. 2003/0195588 (“Fischell”) describes an in-ear stimulator adapted to provide magnetic, electrical, audible, tactile or caloric stimulation. Fischell proposes a ring-shaped caloric transducer strip that may result in relatively slow thermal changes of the external auditory canal.

U.S. Patent Publication Nos. 2011/0313499 and 2011/0313498 disclose in-ear stimulators for administering thermal stimulation to the external auditory canal(s) of a subject, said stimulators comprising an earpiece thermally coupled to a thermoelectric device. Relatively fast temperature changes may be achieved using the thermoelectric device.

Devices, systems and methods for stimulating the nervous and/or vestibular systems of a subject with relatively fast temperature changes are potentially beneficial and may allow users to take full advantage of physiological responses useful in diagnosing and/or treating a variety of medical conditions.

### SUMMARY OF THE INVENTION

A first aspect of the present invention is a system comprising a gas preparation device configured to cool, heat and/or humidify a gas or mixture of gases and one or more conduits configured to deliver the cooled, heated and/or humidified gas(es) to the external auditory canal(s) of a subject.

A second aspect of the present invention is a method comprising administering one or more cooled, heated and/or humidified gases to the external auditory canal(s) of a subject.

The foregoing and other objects and aspects of the present invention are explained in detail in the drawings and specification set forth below.

### BRIEF DESCRIPTION OF THE DRAWINGS

FIGS. 1-7 depict gas preparation devices according to some embodiments of the present invention.

FIGS. 8-10 depict systems according to some embodiments of the present invention.

FIGS. 11-13 are flowcharts of methods according to some embodiments of the present invention.

### DETAILED DESCRIPTION

The present invention is explained in greater detail below. This description is not intended to be a detailed catalog of all the different ways in which the invention may be implemented or of all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein, which do not depart from the instant invention, will be apparent to those skilled in the art in light of the instant disclosure. Hence, the following specification is intended to illustrate some particular embodiments of the invention, and not to exhaustively specify all permutations, combinations and variations thereof.

All patents, patent publications, non-patent publications referenced herein are incorporated by reference in their entireties for all purposes and to the same extent as if each was specifically and individually indicated to be incorporated by reference.

Aspects of the present invention are described below with reference to block diagrams and/or flowchart illustrations of methods, systems and/or computer program products according to embodiments of the invention.

It is to be understood that when an element or layer is referred to as being “on”, “attached to”, “connected to”, “coupled to”, “coupled with” or “contacting” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. It will also be appreciated by those of skill in the art that references to a structure or feature that is disposed “adjacent” another structure or feature may have portions that overlap or underlie the adjacent structure or feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It is to be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

It is to be understood that various blocks of the block diagrams and/or flowchart illustrations, and combinations of blocks in the block diagrams and/or flowchart illustrations, can be implemented by computer program instructions. These computer program instructions may be provided to a processor of a general purpose computer, special purpose computer and/or other programmable data processing apparatus to produce a machine, such that the instructions, which execute via the processor of the computer or other programmable data processing apparatus, create means for implementing the functions/acts specified in the block diagrams and/or flowchart illustrations. The computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagram and/or flowchart illustrations. The computer program instructions may also be loaded onto a computer or other programmable data processing apparatus to cause a series of operational steps to be performed on the computer or other programmable data processing apparatus to produce a computer-implemented process such that the instructions which execute on the computer or other programmable data processing apparatus provide steps for implementing the functions/acts specified in the block diagrams and/or flowchart illustrations.

Accordingly, aspects of present invention may be embodied in hardware and/or software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system. In the context of this document, a computer usable or computer-readable non-transient storage medium may be any medium that can contain and/or store the program for use by or in connection with the instruction execution system, apparatus or device. For example, the computer-usable or computer-readable medium may be an electronic, optical, electromagnetic, infrared or semiconductor system, apparatus or device.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

As used herein, the terms “a” or “an” or “the” may refer to one or more than one unless the context clearly dictates otherwise.

As used herein, the term “and/or” refers to and encompasses any and all possible combinations of one or more of the associated listed items, as well as the lack of combinations when interpreted in the alternative (“or”).

As used herein, the term “about,” when used in reference to a measurable value such as an amount of mass, dose, time, temperature, and the like, refers to a variation of 0.1%, 0.25%, 0.5%, 0.75%, 1%, 2%, 3%, 4%, 5%, 6,%, 7%, 8%, 9%, 10%, 15% or even 20% of the specified amount.

As used herein, the terms “actively controlled waveform” and “actively controlled, time-varying thermal waveform” refer to a thermal waveform in which the intensity and/or the directionality of the activation signal(s) used to deliver the thermal waveform and/or the temperature of the gas(es) used to deliver the thermal waveform is repeatedly adjusted (e.g., continuously adjusted or substantially continuously adjusted) during delivery of the thermal waveform. For example, the activation signal driving the cooling and/or heating elements used to deliver the thermal waveform may be continuously adjusted in response to feedback data from one or more sensors (e.g., a temperature sensor configured to sense the temperature of the gas(es) delivered to a subject's external auditory canal). Such active control may be used to minimize errors in the delivery of a prescribed thermal waveform (e.g., by minimizing thermal drift, which may otherwise allow the subject's body temperature to adversely affect the accuracy of the thermal waveform(s) delivered to a subject′ external auditory canal(s)).

As used herein, the term “adjuvant treatment” refers to a treatment session in which the delivery of one or more thermal stimuli to the external auditory canal(s) of a subject modifies the effect(s) of one or more active agents and/or therapies. For example, the delivery of one or more thermal waveforms, via the external auditory canal(s), to the vestibular system and/or the nervous system of a subject may enhance the effectiveness of a pharmaceutical agent (by restoring the therapeutic efficacy of a drug to which the subject had previously become habituated, for example). Likewise, the delivery of one or more thermal waveforms, via the external auditory canal(s), to the vestibular system and/or the nervous system of a subject may enhance the effectiveness of counseling or psychotherapy. In some embodiments, delivery of one or more thermal stimuli to the external auditory canal(s) of a subject may reduce or eliminate the need for one or more active agents and/or therapies. Adjuvant treatments may be effectuated by delivering one or more thermal waveforms to the external auditory canal(s) of a subject prior to, currently with and/or after administration of one or more active agents and/or therapies.

As used herein, the terms “chronic treatment,” “chronically treating” and the like refer to a therapeutic treatment carried out at least once per week/month (e.g., two or three times per week, daily, etc.) over an extended period of time. Chronic treatment typically lasts at least one to two weeks (and, in some embodiments, at least one to two months), but may last as long as required to achieve and/or maintain therapeutic efficacy for the particular condition or disorder for which the treatment is carried out (i.e., the device may be used periodically throughout the subject's life).

As used herein, the term “sensor feedback data” refers to data associated with one or more characteristics (e.g., temperature, humidity, pressure and/or velocity) of a gas (or mixture of gases) before it enters the gas preparation device, as it enters the gas preparation device, before it has been cooled by the gas preparation device, as it is being cooled by the gas preparation device, after it has been cooled by the gas preparation device, before it has been heated by the gas preparation device, as it is being heated by the gas preparation device, after it has been heated by the gas preparation device, before it has been humidified by the gas preparation device, as it is being humidified by the gas preparation device, after it has been humidified by the gas preparation device, before it has passed from one cooling/heating/humidifying apparatus to another within the gas preparation device, as it passes from one cooling/heating/humidifying apparatus to another within the gas preparation device, after it has passed from one cooling/heating/humidifying apparatus to another within the gas preparation device, before it exits the gas preparation device, as it exits the gas preparation device and/or after it exits the gas preparation device (e.g, as it passes through a conduit operatively connected to the gas preparation device and/or as it exits a conduit operatively connected to the gas preparation device. Sensor feedback data may be used to verify that gas(es) of the appropriate temperature(s), humidity(ies), pressure(s), velocity(ies), etc. is/are being generated by the gas preparation device (or a component thereo) and/or delivered to the external auditory canal(s) of the subject, to enable/cause a controller to increase/decrease the activation of one or more cooling apparatuses, one or more heating apparatuses and/or one or more humidifying apparatuses to ensure that gas(es) of the appropriate temperature(s)/humidity(ies) is/are being generated by the gas preparation device and/or delivered to the external auditory canal(s) of a subject, and/or to trigger a system shutdown if one or more characteristics of the gas(es) approaches and/or crosses a safety threshold (e.g., if the temperature(s) of the gas(es) drop(s) below a low temperature threshold (e.g., about 10 degrees Centigrade) or exceed(s) a high temperature threshold (e.g., about 50 degrees Centigrade)).

As used herein, the term “data associated with the delivery of one or more thermal waveforms” refers to information associated with the delivery of one or more thermal waveforms and may include, but is not limited to, data associated with the target time/temperature parameters of the thermal waveform(s), the time/temperature parameters of the thermal waveform(s) delivered; the date/time of delivery of the thermal waveform(s), the temperature of the subject's external auditory canal(s) at various time points before, during and/or after delivery of the thermal waveform(s); the temperature of the subject's inner ear(s) at various time points before, during and/or after delivery of the thermal waveform(s); subject-specific time constants (e.g., a time constant associated with the transduction of heat from the external auditory canal to the inner ear); reaction time (i.e., how long it took for the subject to react to the thermal waveform(s)); effectiveness of the thermal waveform(s) (e.g., whether and to what extent symptoms were relieved, whether the thermal waveform(s) enhanced the effectiveness of another agent/therapy, etc.); stability of the treatment (i.e., how long the effects of the treatment lasted); instability of the treatment (i.e., which condition(s) and/or symptom(s) returned and when did it/they return); the presence or absence of comorbid disorders, injuries and/or diseases; disorder, injury and/or disease modulation(s) and/or other modification(s) that occurred as a result of treatment; the cognitive effect(s) of one or more thermal waveforms; subject compliance (e.g., whether the subject initiated delivery at the prescribed time, whether the subject completed the prescribed treatment session, etc.); the mood of the subject before, during and/or after his/her treatment session(s) (e.g., videos/images of a subject that may be used to assess mood); objectives measures of efficacy (e.g., nystamography data, EEG data, MRI data, heart rate data, blood pressure data, transcranial Doppler (TCD) data (e.g., functional TCD sonography and/or functional TCD spectroscopy)); subjective measures of efficacy (e.g., a subject-reported pain score); blood chemistry data (e.g., blood A1c levels, blood glucose levels and blood cortisol levels); saliva chemistry data (e.g., saliva cortisol levels); urine chemistry data (e.g., urine cortisol levels)) and comments the subject made about his/her treatment session(s) (e.g., comments made to a physician, submitted in response to an automated survey and/or recorded in a treatment diary). In some embodiments, data associated with the delivery of one or more thermal waveforms comprises sensor feedback data, subject feedback data and/or physician feedback data. In some embodiments, data associated with the delivery of one or more thermal waveforms comprises, consists essentially of or consists of data associated with the precise time/temperature parameters of the thermal waveform(s) delivered to the subject and a subjective measure of efficacy (e.g., a subject-reported pain score).

As used herein, the terms “idealized thermal waveform” and “idealized waveform” refer to a thermal waveform that has been indicated and/or approved for use in the treatment of one or more diseases/disorders/injuries and/or for use in the provision of neuroprotection, enhanced cognition and/or increased cognitive reserve. For example, a thermal waveform may be indicated for use in the treatment of migraines if it has effectively treated migraines in the past or if it belongs to a class of thermal waveforms that are known to treat migraines. Likewise, a thermal waveform may be approved for use in the treatment of a given disorder if it has received regulatory approval (e.g. FDA approval) for such use, or if it belongs to a class of thermal waveforms that have been approved for the treatment of that disorder. An idealized thermal waveform may be indicated/approved for use in the treatment of multiple diseases/disorders/injuries.

As used herein, the term “physician feedback data” refers to data associated with physician feedback regarding the delivery of one or more thermal waveforms. Physician feedback data may comprise, but is not limited to, subject information from the subject history database of one or more physician control devices and comments from one or more physicians (e.g., comments regarding a physician's opinion as to the efficacy of a given waveform or the effect(s) of certain waveform modifications, etc.).

As used herein, the terms “prescription” and “prescription protocol” refer to a set of instructions and/or limitations associated with thermal stimulation of the vestibular system and/or the nervous system of a subject via the external auditory canal(s) of the subject. In some embodiments, a prescription comprises, consists essentially of or consists of a set of instructions for administering one or more thermal waveforms (e.g., one or more actively controlled, time-varying thermal waveforms) to the vestibular system and/or the nervous system of a subject (e.g., by delivering one or more cooled, heated and/or humidified gases (or mixtures of gases) to the external auditory canal(s) of the subject). A prescription may comprise a set of instructions for delivering thermal stimulation to the left vestibular system of a subject by delivering one or more thermal waveforms (e.g., one or more idealized thermal waveforms) to the left external auditory canal of the subject and/or a set of instructions for delivering thermal stimulation to the right vestibular system of a subject by delivering one or more thermal waveforms (e.g., one or more idealized thermal waveforms) to the right external auditory canal of the subject (i.e., one prescription may comprise instructions for stimulating both the right and left vestibular systems of a subject). A prescription may comprise any suitable instructions and/or limitations, including, but not limited to, the parameters of the waveform(s) to be delivered to the subject, the number and frequency of treatment sessions (e.g., X treatment sessions over Y time period), a limitation as to how many treatment sessions may be administered during a given time period (e.g., no more than X treatment sessions within Y time period), instructions as to which thermal waveform(s) will be administered during a given treatment session (and in what order they are to be administered), instructions as to which vestibular system will receive a given waveform (e.g., right, left or both) and an expiration date. In some embodiments, a prescription comprises instructions for delivering a placebo (i.e., for fooling a subject into believing that one or more thermal waveforms has been delivered even though no such deliver has occurred and/or that a particular thermal waveform (or set of waveforms) has been delivered even though a different thermal waveform (or set of waveforms) was actually delivered). In some embodiments, the prescription is generated by a physician. Any conventional security means may be provided to prevent unauthorized modification of the prescription (e.g., the prescription may be password protected, with only the prescribing physician having knowledge of and/or access to the password).

As used herein, the terms “prevent,” “prevention,” and “preventing” refer to delaying and/or inhibiting the onset of a disease or disorder as described herein, or at least one symptom of a disease or disorder as described herein (e.g., inhibiting the onset of tremors, bradykinesia, rigidity and/or postural instability associated with Parkinson's disease; inhibiting the onset of one or more intrusive symptoms (e.g., dissociative states, flashbacks, intrusive emotions, intrusive memories, nightmares, and night terrors), one or more avoidant symptoms (e.g., avoiding emotions, avoiding relationships, avoiding responsibility for others, avoiding situations reminiscent of the traumatic event) and/or one or more hyperarousal symptoms (e.g., exaggerated startle reaction, explosive outbursts, extreme vigilance, irritability, panic symptoms, sleep disturbance) associated with post-traumatic stress disorder). In some embodiments, prevention of one or more symptoms of a disease or disorder occurs after the onset of one or more other symptoms of the disease or disorder. In some embodiments, prevention occurs in the absence of symptoms. For example, one or more symptoms of a disease or disorder may be prevented by administering one or more thermal waveforms (e.g., one or more idealized thermal waveforms) to the external auditory canal(s) of a susceptible individual prior to the onset of symptoms (e.g., in light of a history of symptoms and/or in light of genetic or other susceptibility factors). In some embodiments, prevention occurs after one or more symptoms have resolved—for example, to prevent or delay their recurrence.

As used herein, the term “subject” refers to both human subjects and animal subjects, including, but not limited to, mice, rats, rabbits, cats, dogs, pigs, horses, monkeys, apes, etc. The subject may be male or female. That subject may be of any suitable age, including infant, juvenile, adolescent, adult and geriatric ages. In some embodiments, the methods, devices and systems of the present invention may be used to induce physiological and/or psychological responses in a subject for medically diagnostic and/or therapeutic purposes. For example, the methods, devices and systems of the present invention may be used to diagnose and/or treat mammalian subjects, such as mice, rats, pigs and monkeys, for medical research or veterinary purposes.

As used herein, the term “subject information” refers to data associated with one or more subjects. Subject information may comprise, but is not limited to, information related to a subject's identity, a subject's cognitive abilities, a subject's medical history, a subject's current symptoms (if any), a subject's present diagnosis (if any), a subject's current prescriptions (if any) and data associated with the delivery of one or more thermal waveforms to the vestibular system and/or the nervous system of a subject.

As used herein, the term “subject feedback data” refers to data associated with subject feedback regarding the delivery of one or more thermal waveforms. Subject feedback data may comprise, but is not limited to, a subject's evaluation of their pain level before, during and/or after delivery of the thermal waveform(s) (e.g., subject-reported pain scores given before, during and after a treatment session) and subject comments (e.g., comments regarding a subject's opinion as to the efficacy of a given waveform or the effect(s) of certain waveform modifications, etc.).

As used herein, the terms “treat,” “treatment,” and “treating” refer to reversing, alleviating, reducing the severity of and/or inhibiting the progress of a disease or disorder as described herein, or at least one symptom of a disease or disorder as described herein (e.g., treating one or more of tremors, bradykinesia, rigidity or postural instability associated with Parkinson's disease; treating one or more of intrusive symptoms (e.g., dissociative states, flashbacks, intrusive emotions, intrusive memories, nightmares, and night terrors), avoidant symptoms (e.g., avoiding emotions, avoiding relationships, avoiding responsibility for others, avoiding situations reminiscent of the traumatic event) or hyperarousal symptoms (e.g., exaggerated startle reaction, explosive outbursts, extreme vigilance, irritability, panic symptoms, sleep disturbance) associated with post-traumatic stress disorder). In some embodiments, treatment is administered after one or more symptoms have developed. In other embodiments, treatment is administered in the absence of symptoms. For example, treatment may be administered to a susceptible individual prior to the onset of symptoms (e.g., in light of a history of symptoms and/or in light of genetic or other susceptibility factors). In some embodiments, treatment is continued after symptoms have resolved—for example, to prevent or delay their recurrence. Treatment may comprise providing neuroprotection, enhancing cognition and/or increasing cognitive reserve. Treatment may be as an adjuvant treatment as further described herein.

As used herein, the term “vestibular system” has the meaning ascribed to it in the medical arts and includes, but is not limited to, those portions of the inner ear known as the vestibular apparatus and the vestibulocochlear nerve. The vestibular system, therefore, further includes, but is not limited to, those parts of the brain that process signals from the vestibulocochlear nerve (e.g., brain stem, cerebellum, hypothalamus, hippocampus, cerebral cortex).

As used herein, the terms “waveform,” “waveform stimulus” and “thermal waveform” refer to a thermal stimulus (heating and/or cooling) delivered to the external auditory canal of a subject. “Waveform” is not to be confused with “frequency,” the latter term concerning the rate of delivery of a particular waveform. The term “waveform” is used herein to refer to one complete cycle thereof, unless additional cycles (of the same, or different, waveform) are indicated. As discussed further below, time-varying thermal waveforms are preferred over square waveforms in carrying out the present invention.

In general, a waveform of the present invention comprises a leading edge, a peak, and a trailing edge.

The waveform leading edge is preferably ramped or time-varying: that is, the amplitude of the waveform increases through a plurality of different temperature points over time (e.g., at least 5, 10, or 15 or more distinct temperature points, and in some embodiments at least 50, 100, or 150 or more distinct temperature points, from start to peak). The shape of the leading edge may be a linear ramp, a curved ramp (e.g., convex or concave; logarithmic or exponential), or a combination thereof. A vertical cut may be included in the waveform leading edge, so long as the remaining portion of the leading edge progresses through a plurality of different temperature points over time as noted above.

The peak of the waveform represents the amplitude of the waveform as compared to the subject's body temperature. In general, an amplitude of at least 5 or 7 degrees Centigrade is preferred for both heating and cooling waveform stimulation. In general, an amplitude of up to 25 degrees Centigrade is preferred for cooling waveform stimulation (e.g., 15 degrees Centigrade). In general, an amplitude of up to 8 or 10 degrees Centigrade is preferred for heating waveform stimulus (e.g., 6 degrees Centigrade). The peak of the waveform may be truncated (that is, the waveform may reach an extended temperature plateau), as long as the desired characteristics of the leading edge, and preferably trailing edge, are retained. For heating waveforms, truncated peaks of long duration (that is, maximum heat for a long duration) are less preferred, particularly at higher heats, because of the potential that the subject may experience a burning sensation.

The waveform trailing edge is preferably ramped or time-varying: that is, the amplitude of the waveform decreases through a plurality of different temperature points over time (e.g., at least 5, 10, or 15 or more distinct temperature points, or in some embodiments at least 50, 100, or 150 or more distinct temperature points, from peak to trough). The shape of the trailing edge may be a linear ramp, a curved ramp (e.g., convex or concave; logarithmic or exponential), or a combination thereof. A vertical cut may again be included in the waveform trailing edge, as long as the remaining portion of the trailing edge progresses through a plurality of different temperature points over time as noted above.

The duration of a waveform (or the frequency of that waveform stimulus) is the time from the onset of the leading edge to either the conclusion of the trailing edge or (in the case of a vertically cut waveform) the onset of a subsequent waveform. In general, each waveform has a duration, or frequency, of from one or two minutes up to ten or twenty minutes.

If a first waveform is followed by a second waveform, the minimal stimulus point therebetween (i.e., the point of minimal heating or minimal cooling) is referred to as a trough. Like a peak, the trough may be truncated, as long as the desired characteristics of the trailing edge and the following leading edge are retained. While the trough may represent a return to the subject's current body temperature, in some embodiments minor thermal stimulation (e.g., cooling or heating by 1 or 2 degrees up to 4 or 5 degrees Centigrade) may continue to be applied at the trough (or through a truncated trough).

In any given testing/treatment session, a plurality of waveforms may be delivered in sequence. In general, a testing/treatment session will comprise 1, 2, 3, 4, 5, 6, 7, 8, 9, 10, 11, 12, 13, 14, 15, 16, 17, 18, 19, 20 waveforms or more delivered sequentially. Each individual waveform may be the same, or different, from the other waveform(s).

The first waveform of a testing/treatment session is initiated at a start point, which start point may be at or about the subject's body temperature at the time the treatment session is initiated (typically a range of about 34 to 38 degrees Centigrade, around a normal body temperature of about 37 degrees Centigrade. The lower point, 34, is due to the coolness of the external auditory canal(s) in comparison with the rest of the body. It typically will not be above about 37 unless the subject is febrile). Note that, while the subject's external auditory canal(s) may be slightly less than body temperature (e.g., about 34 to 36 degrees Centigrade), the starting temperature for the thermal waveform is typically body temperature (the temperature of the inner ear), or about 37 degrees Centigrade. In some embodiments, however, the temperature of the conduit(s) may not have equilibrated with the external auditory canal(s) prior to the start of the testing/treatment session, and in such case the start point for at least the first waveform may be at a value closer to room temperature (normally bout 23 to 26 degrees Centigrade).

A testing/treatment session may have a total duration of 5, 10, 15, 20 25, 30, 35, 40, 45, 50, 55, 60, 90, 120 minutes or more, depending on factors such as the specific waveform(s) delivered, the subject, the condition(s) being treated, the benefit(s) being sought, etc. In some embodiments, a testing/treatment session may comprise one or more breaks (i.e., periods wherein no thermal stimulus is delivered). For example, a testing/treatment session may comprise multiple stimulation periods with a break between each stimulation period and may have a total duration of 60 minutes or more.

Testing/treatment sessions are preferably once a day, though in some embodiments more frequent testing/treatment sessions (e.g., two or three times a day or more) may be employed. Day-to-day testing/treatment sessions may be by any suitable schedule: every day, every other day, twice a week, as needed, etc.

The present invention provides gas preparation devices for cooling, heating and/or humidifying a gas (or a mixture of gases). In some embodiments, the gas preparation device comprises, consists essentially of or consists of one or more gas cooling apparatuses configured to cool the gas(es), one or more gas heating apparatuses configured to heat the gas(es) and/or one or more gas humidifying apparatuses configured to humidify the gas(es). In some embodiments, the gas preparation device comprises one or more controllers configured to control the degree to which the gas(es) is/are cooled, heated and/or humidified. In some embodiments, the gas preparation device comprises one of more sensors configured to detect one or more characteristics of the gas(es) and to provide feedback to the controller(s), which may utilize that feedback to modulate the degree to which gas(es) is/are cooled, heated and/or humidified. In some embodiments, the gas preparation device comprises a gas purifier (e.g., a gas filtration device/system configured to remove particulate matter and/or volatile chemicals, to kill gasborne microbes, etc.) and/or a gas compressor.

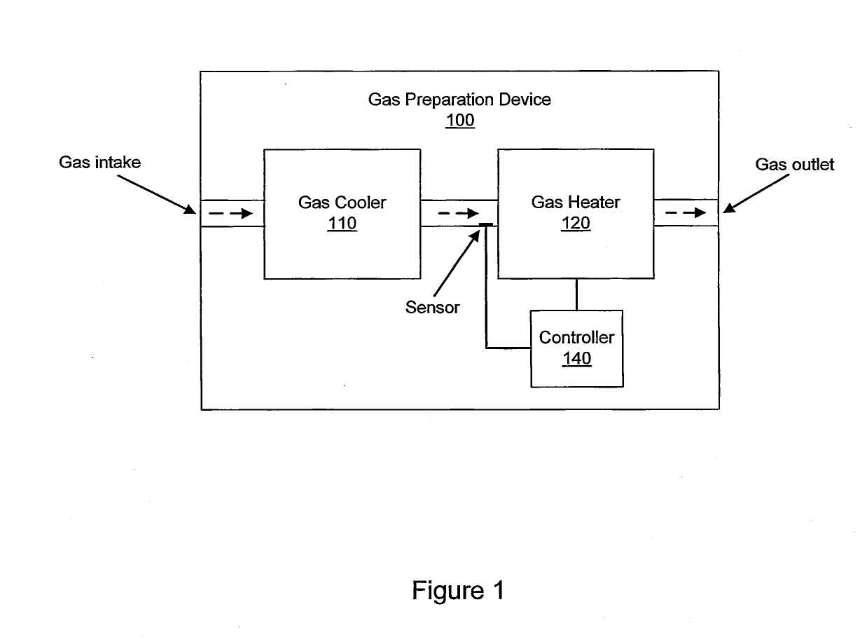

As shown in FIG. 1, in some embodiments, the gas preparation device 100 comprises a gas intake, a gas cooler 110, a conduit for transferring a gas (or mixture of gases) from the gas intake to the gas cooler 110, a gas heater 120, a conduit for transferring a gas (or mixture of gases) from the gas cooler 110 to the gas heater 120, a gas outlet, and a conduit for transferring a gas (or mixture of gases) from the gas heater 120 to the gas outlet. A controller 140 is operatively connected to both the gas heater 120 and a sensor located in the conduit carrying the gas(es) from the gas cooler 110 to the gas heater 120 and is configured to activate the gas heater 120 responsive to sensor feedback data from the sensor (e.g., information associated with the temperature(s) of the gas(es) flowing through the conduit).

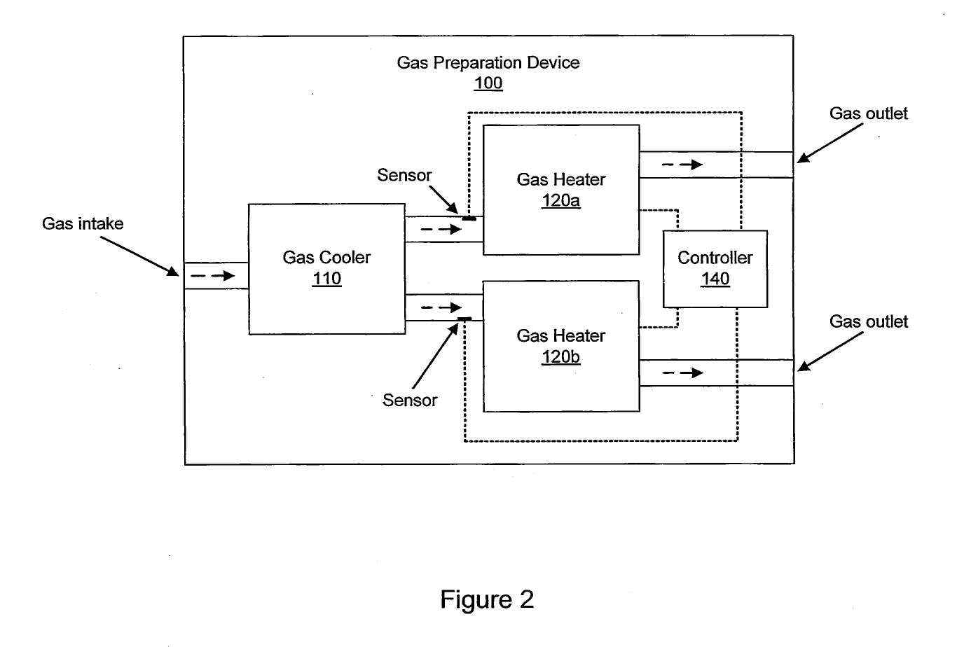

As shown in FIG. 2, in some embodiments, the gas preparation device 100 comprises a gas intake, a gas cooler 110, a first gas heater 120a, a first conduit for transferring a gas (or mixture of gases) from the gas cooler 110 to the first gas heater 120a, a second gas heater 120b, a second conduit for transferring a gas (or mixture of gases) from the gas cooler 110 to the second gas heater 120b, two gas outlets, and conduits for transferring a gas (or mixture of gases) from each of the first and second gas heaters 120a, 120b to one of the gas outlets. A controller 140 is operatively connected to each of the first and second gas heaters 120a, 120b and to sensors located in the first and second conduits. The controller 140 is configured to activate the first gas heater 120a responsive to sensor feedback data from the sensor located in the first conduit (e.g., information associated with the temperature of the gas(es) flowing through the first conduit) and to activate the second gas heater 120b responsive to sensor feedback data from the sensor located in the second conduit (e.g., information associated with the temperature of the gas(es) flowing through the second conduit). Thus, the gas preparation device 100 may be configured to generate a first time-varying thermal waveform using the gas cooler 110 and the first gas heater 120a and a second time-varying thermal waveform using the gas cooler 110 and the second gas heater 120b.

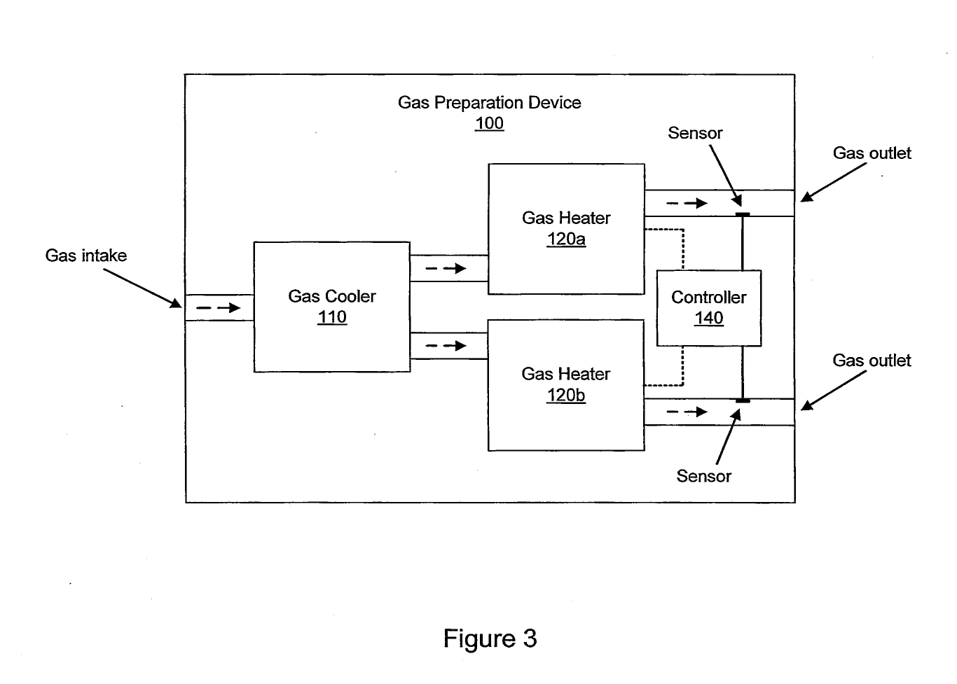

As shown in FIG. 3, in some embodiments, the gas preparation device 100 comprises a gas intake, a gas cooler 110, a first gas heater 120a, a first conduit for transferring a gas (or mixture of gases) from the gas cooler 110 to the first gas heater 120a, a second gas heater 120b, a second conduit for transferring a gas (or mixture of gases) from the gas cooler 110 to the second gas heater 120b, two gas outlets, and conduits for transferring a gas (or mixture of gases) from each of the first and second gas heaters 120a, 120b to one of the gas outlets. A controller 140 is operatively connected to each of the first and second gas heaters 120a, 120b and to sensors located in the conduits carrying gas(es) from the first and second gas heaters 120a, 120b. The controller 140 is configured to activate the first gas heater 120a responsive to sensor feedback data from the sensor located in the conduit carrying gas(es) from the first gas heater 120a (e.g., information associated with the temperature of the gas(es) flowing from the first gas heater 120a) and to activate the second gas heater 120b responsive to sensor feedback data from the sensor located in the conduit carrying gas(es) from the second gas heater 120b (e.g., information associated with the temperature of the gas(es) flowing from the second gas heater 120b). Thus, the gas preparation device 100 may be configured to generate a first time-varying thermal waveform using the gas cooler 110 and the first gas heater 120a and a second time-varying thermal waveform using the gas cooler 110 and the second gas heater 120b.

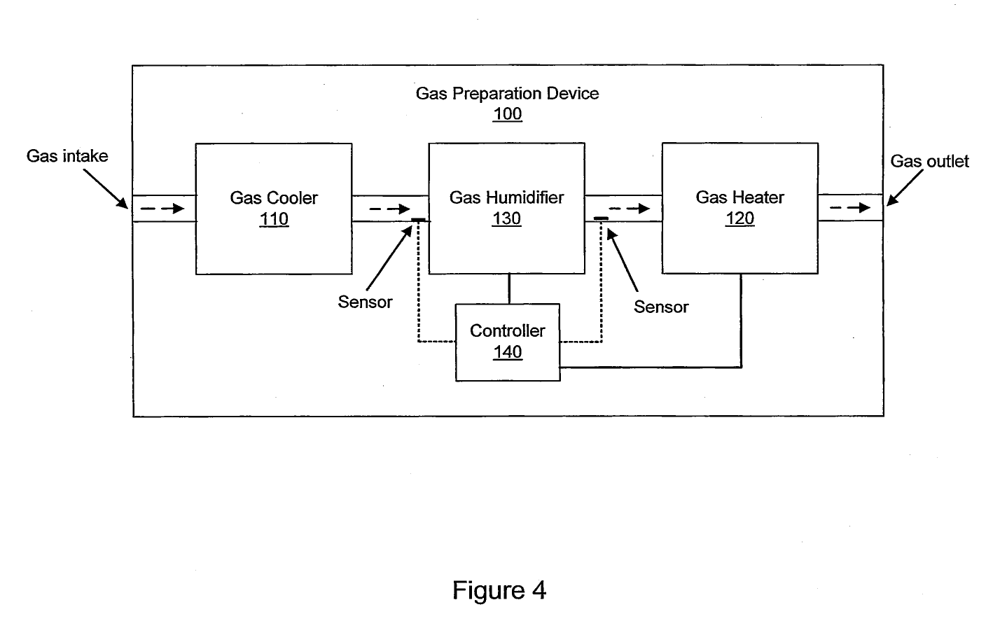

As shown in FIG. 4, in some embodiments, the gas preparation device 100 comprises a gas intake, a gas cooler 110, a gas humidifier 130, a first conduit for transferring a gas (or mixture of gases) from the gas cooler 110 to the gas humidifier 130, an gas heater 120, a second conduit for transferring a gas (or mixture of gases) from the gas humidifier 130 to the gas heater 120, a gas outlet, and a conduit for transferring a gas (or mixture of gases) from the gas heater 120 to the gas outlet. A controller 140 is operatively connected to the gas heater 120, to the gas humidifier 130 and to sensors located in the first and second conduits. The controller 140 is configured activate the gas humidifier 130 responsive to sensor feedback data from the sensor located in the first conduit (e.g., information associated with the humidity of the gas(es) flowing through the first conduit) and to activate the gas heater 120 responsive to sensor feedback data from the sensor located in the second conduit (e.g., information associated with the temperature of the gas(es) flowing through the second conduit).

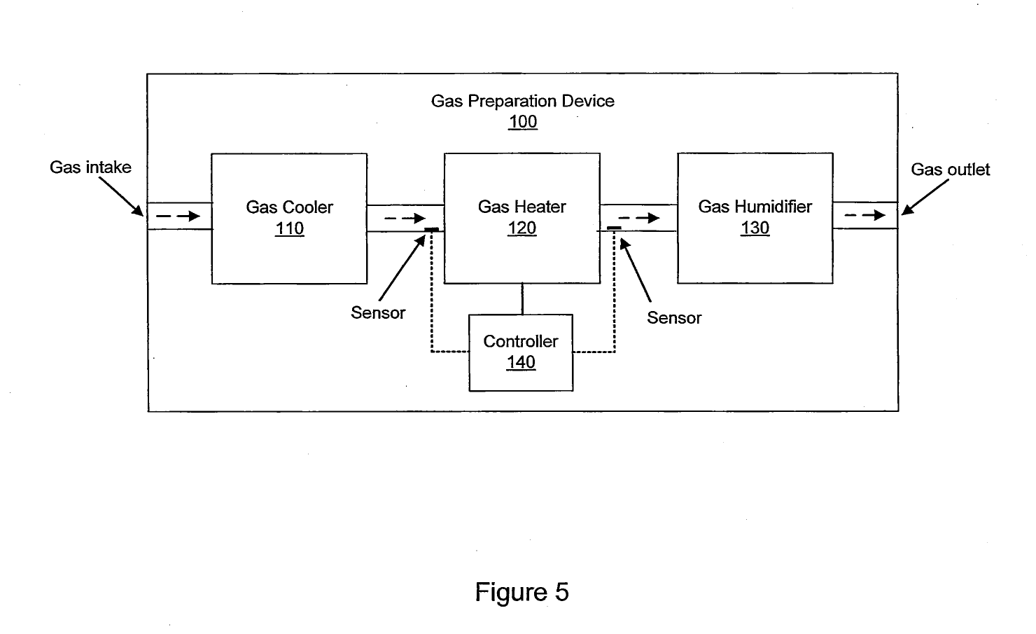

As shown in FIG. 5, in some embodiments, the gas preparation device 100 comprises a gas intake, a gas cooler 110, a gas heater 120, a first conduit for transferring a gas (or mixture of gases) from the gas cooler 110 to the gas heater 120, a gas humidifier 130, a second conduit for transferring a gas (or mixture of gases) from the gas heater 120 to the gas humidifier 130, a gas outlet, and a conduit for transferring a gas (or mixture of gases) from the gas humidifier 130 to the gas outlet. A controller 140 is operatively connected to both the gas heater 120 and to sensors located in the first and second conduits. The controller 140 is configured activate the gas heater 120 responsive to sensor feedback data from the sensors located in the first and second conduits (e.g., information associated with the temperature of the gas(es) flowing through the first and second conduits).

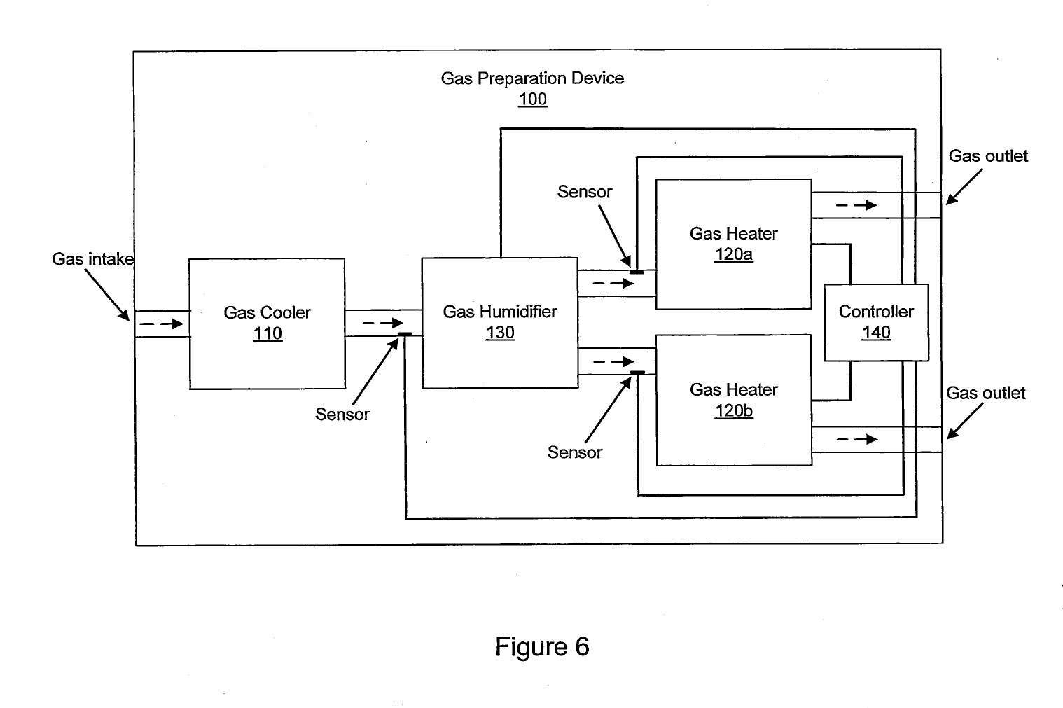

As shown in FIG. 6, in some embodiments, the gas preparation device 100 comprises a gas intake, a gas cooler 110, a gas humidifier 130, a third conduit for transferring a gas (or mixture of gases) from the gas cooler 110 to the gas humidifier 130, a first gas heater 120a, a first conduit for transferring a gas (or mixture of gases) from the gas humidifier 130 to the first gas heater 120a, a second gas heater 120b, a second conduit for transferring a gas (or mixture of gases) from the gas humidifier 130 to the second gas heater 120b, two gas outlets, and conduits for transferring a gas (or mixture of gases) from each of the first and second gas heaters 120a, 120b to one of the gas outlets. A controller 140 is operatively connected to the gas humidifier 130, to the first and second gas heaters 120a, 120b and to sensors located in the first, second and third conduits. The controller 140 is configured to activate the first gas heater 120a responsive to sensor feedback data from the sensor located in the first conduit (e.g., information associated with the temperature of the gas(es) flowing through the first conduit), to activate the second gas heater 120b responsive to sensor feedback data from the sensor located in the second conduit (e.g., information associated with the temperature of the gas(es) flowing through the second conduit), and to activate the gas humidifier 130 responsive to sensor feedback data from the sensor located in the third conduit (e.g., information associated with the humidity of the gas(es) flowing through the third conduit). Thus, the gas preparation device 100 may be configured to generate a first time-varying thermal waveform using the gas cooler 110, the gas humidifier 130 and the first gas heater 120a and a second time-varying thermal waveform using the gas cooler 110, the gas humidifier 130 and the second gas heater 120b.

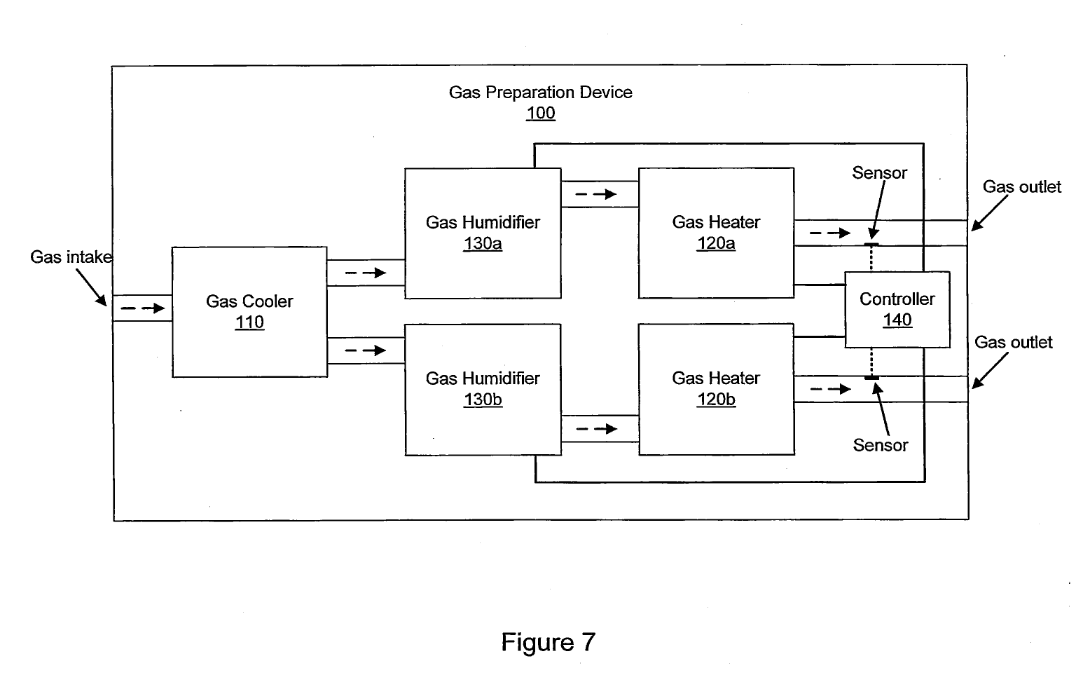

As shown in FIG. 7, in some embodiments, the gas preparation device 100 comprises a gas intake, a gas cooler 110, a first gas humidifier 130a, a conduit for transferring a gas (or mixture of gases) from the gas cooler 110 to the first gas humidifier 130a, a first gas heater 120a, a conduit for transferring a gas (or mixture of gases) from the first gas humidifier 130a to the first gas heater 120a, a second gas humidifier 130b, a conduit for transferring a gas (or mixture of gases) from the gas cooler 110 to the second gas humidifier 130b, a second gas heater 120b, a conduit for transferring a gas (or mixture of gases) from the second gas humidifier 130a to the second gas heater 120b, two gas outlets, and conduits for transferring a gas (or mixture of gases) from each of the first and second gas heaters 120a, 120b to one of the gas outlets. A controller 140 is operatively connected to the first and second gas humidifiers 130a, 130b, to the first and second gas heaters 120a, 120b and to sensors located in the conduits carrying gas(es) from the first and second gas heaters 120a, 120b. The controller 140 is configured to activate the first gas humidifier 130a and the first gas heater 120a responsive to sensor feedback data from the sensor located in the conduit carrying gas(es) from the first gas heater 120a (e.g., information associated with the temperature and humidity of the gas(es) flowing from the first gas heater 120a) and to activate the second gas humidifier 130b and the second gas heater 120b responsive to sensor feedback data from the sensor located in the conduit carrying gas(es) from the second gas heater 120b (e.g., information associated with the temperature and humidity of the gas(es) flowing from the second gas heater 120b). Thus, the gas preparation device 100 may be configured to generate a first time-varying thermal waveform using the gas cooler 110, the first gas humidifier 130a and the first gas heater 120a and a second time-varying thermal waveform using the gas cooler 110, the second gas humidifier 130b and the second gas heater 120b.

Gas preparation devices of the present invention may comprise any suitable gas intake(s) and may receive gas(es) from any suitable source(s), including, but not limited to, gas purifiers (e.g., gas filtration devices/systems configured to remove particulate matter and/or volatile chemicals, to kill gasborne microbes, etc.), gas humidifiers, gas dehumidifiers, gas compressors, gas lines (e.g., an oxygen supply line in a hospital) and gas storage vessels (e.g., gas canisters). In some embodiments, the gas preparation device utilizes ambient air that has been purified, humidified, dehumidified and/or compressed. In some embodiments, the gas preparation device utilizes a gas (or mixture of gases) from a storage vessel (e.g., a refillable or disposable gas canister comprising one or more pressurized gases).

Gas preparation devices of the present invention may comprise any suitable gas outlet(s) and may transfer gas(es) to any suitable device(s), including, but not limited to, one or more external conduits (e.g., one or more conduits configured to deliver the cooled, heated and/or humidified gas(es) to the external auditory canal(s) of a subject).

Gas preparation devices of the present invention may be configured to cool, heat and/or humidify gas(es) of any suitable flow rate(s). In some embodiments, the gas (or mixture of gases) has a flow rate in the range of about 1 to about 20 liters per minute or more (e.g., about 1, 2, 3, 4, 5, 6, 7, 8, 9, 10, 11, 12, 13, 14, 15, 16, 17, 18, 19, 20 liters per minute or more). In some embodiments, the gas (or mixture of gases) has a flow rate in the range of about 5 to about 10 liters per minute or more (e.g., about 5, 6, 7, 8, 9, 10 liters per minute or more).

Gas preparation devices of the present invention may be configured to cool and/or heat gas(es) to any suitable temperature(s). In some embodiments, the gas preparation device is configured to cool and/or heat a gas (or mixture of gases) such that it has a temperature in the range of about 1 to about 60 degrees Centigrade (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 degrees Centigrade). In some embodiments, the gas preparation device is configured to cool and/or heat a gas (or mixture of gases) such that it has a temperature in the range of about 5 to about 55 degrees Centigrade (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 degrees Centigrade). In some embodiments, the gas preparation device is configured to cool and/or heat a gas (or mixture of gases) such that it has a temperature in the range of about 10 to about 50 degrees Centigrade (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees Centigrade).

Gas preparation devices of the present invention may comprise any suitable gas cooling apparatus(es). In some embodiments, the gas preparation device comprises one or more vapor-compression refrigeration elements, one or more vapor-absorption refrigeration elements, one or more magnetic refrigeration elements and/or one or more thermoelectric cooling elements.

The gas cooling apparatus(es) may be configured to cool gas(es) to any suitable temperature(s). In some embodiments, the gas cooling apparatus is configured to cool a gas (or mixture of gases) to a temperature in the range of about 1 to about 60 degrees Centigrade (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 degrees Centigrade). In some embodiments, the gas cooling apparatus is configured to cool a gas (or mixture of gases) to a temperature in the range of about 5 to about 55 degrees Centigrade (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 degrees Centigrade). In some embodiments, the gas cooling apparatus is configured to cool a gas (or mixture of gases) to a temperature in the range of about 10 to about 50 degrees Centigrade (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees Centigrade).

Gas preparation devices of the present invention may comprise any suitable gas heating apparatus(es). In some embodiments, the gas preparation device comprises one or more resistive heating elements and/or one or more thermoelectric heating elements.

The gas heating apparatus(es) may be configured to heat gas(es) to any suitable temperature(s). In some embodiments, the gas heating apparatus is configured to heat a gas (or mixture of gases) to a temperature in the range of about 1 to about 60 degrees Centigrade (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 degrees Centigrade). In some embodiments, the gas heating apparatus is configured to heat a gas (or mixture of gases) to a temperature in the range of about 5 to about 55 degrees Centigrade (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 degrees Centigrade). In some embodiments, the gas heating apparatus is configured to heat a gas (or mixture of gases) to a temperature in the range of about 10 to about 50 degrees Centigrade (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees Centigrade).

Gas preparation devices of the present invention may comprise any suitable gas humidifying apparatus(es). In some embodiments, the gas preparation device comprises an evaporative humidifier element, a natural humidifier element, a vaporizer element (e.g., a steam humidifier element), an impeller element (e.g., a cool mist humidifier element), an ultrasonic humidifier element and/or a forced-gas humidifier element (e.g., a bypass flow-through humidifier element).

The gas humidifier(s) may be configured to humidify gas(es) to any suitable humidity. In some embodiments, the gas humidifier is configured to humidify a gas (or mixture of gases) to a humidity in the range about 5 to about 95 percent relative/specific humidity (e.g., about 5, 6, 7, 8, 9, 10, 11, 12, 13, 14, 15, 16, 17, 18, 19, 20, 25, 30, 35, 40, 45, 50, 55, 60, 65, 70, 75, 76, 77, 78, 79, 80, 81, 82, 83, 84, 85, 86, 87, 88, 89, 90, 91, 92, 93, 94 or 95 percent relative/specific humidity). In some embodiments, the gas humidifier is configured to humidify a gas (or mixture of gases) to a humidity in the range about 10 to about 90 percent relative/specific humidity (e.g., about 10, 11, 12, 13, 14, 15, 16, 17, 18, 19, 20, 25, 30, 35, 40, 45, 50, 55, 60, 65, 70, 75, 76, 77, 78, 79, 80, 81, 82, 83, 84, 85, 86, 87, 88, 89 or 90 percent relative/specific humidity).

Gas preparation devices of the present invention may cool, heat and/or humidify any suitable gases or mixture of gases, including, but not limited to, oxygen, carbon dioxide, nitrogen, helium, neon, argon, krypton, xenon and combinations thereof. In some embodiments, the gas preparation device is configured to cool, heat and/or humidify ambient air (e.g., ambient air that has been purified, humidified, dehumidified and/or compressed).

Gas preparation devices of the present invention may comprise any suitable controller(s). In some embodiments, the gas preparation device comprises at least one controller that is operatively connected to and/or is part of a gas cooling apparatus and is configured to control the degree to which the gas cooling apparatus cools a gas (or mixture of gases). In some embodiments, the gas preparation device comprises at least one controller that is operatively connected to and/or is part of a gas heating apparatus and is configured to control the degree to which the gas heating apparatus heats a gas (or mixture of gases). In some embodiments, the gas preparation device comprises at least one controller that is operatively connected to and/or is part of a gas humidifying apparatus and is configured to control the degree to which the gas humidifying apparatus humidifies a gas (or mixture of gases).

As will be understood by those skilled in the art, a single controller may be operatively connected to more than one gas cooling apparatus, more than one gas heating apparatus and/or more than one gas humidifying apparatus. In some embodiments, the gas preparation device comprises a first controller that is operatively connected to and configured to control a first set of gas cooling/heating/humidifying apparatuses and a second controller that is operatively connected to and configured to control a second set of gas cooling/heating/humidifying apparatuses. For example, in some embodiments, the gas preparation device comprises a first controller and a second controller, wherein the first controller is operatively connected to a first set of gas cooling/heating/humidifying apparatuses that transmits cooled/heated/humidified gas(es) to a first gas output (e.g., a first gas conduit configured to deliver a gas (or mixture of gases) to the left external auditory canal of a subject) and is configured to control the degree to which the first set of gas cooling/heating/humidifying apparatuses cools/heats/humidifies the gas(es) transmitted to the first conduit and wherein the second controller is operatively connected to a second set of gas cooling/heating/humidifying apparatuses that transmits cooled/heated/humidified gas(es) to a second gas output (e.g., a second gas conduit configured to deliver a gas (or mixture of gases) to the right external auditory canal of a subject) and is configured to control the degree to which the second set of gas cooling/heating/humidifying apparatuses cools/heats/humidifies the gas(es) transmitted to the second conduit. In some embodiments, the gas preparation device comprises a single controller that is operatively connected to a first set of gas cooling/heating/humidifying apparatuses and a second set of gas cooling/heating/humidifying apparatuses and that is configured to selectively and separately activate the first and second sets of gas cooling/heating/humidifying apparatuses. For example, in some embodiments, the gas preparation device comprises a single controller that is operatively connected to both a first set of gas cooling/heating/humidifying apparatuses that transmits cooled/heated/humidified gas(es) to a first gas output (e.g., a first gas conduit configured to deliver a gas (or mixture of gases) to the left external auditory canal of a subject) and a second set of gas cooling/heating/humidifying apparatuses that transmits cooled/heated/humidified gas(es) to a second gas output (e.g., a first second conduit configured to deliver a gas (or mixture of gases) to the right external auditory canal of a subject) and that is configured to selectively and separately control the degree to which the first set of gas cooling/heating/humidifying apparatuses cools/heats/humidifies the gas(es) transmitted to the first conduit and the degree to which the second set of gas cooling/heating/humidifying apparatuses cools/heats/humidifies the gas(es) transmitted to the second conduit. In some such embodiments, the first and second sets of gas cooling/heating/humidifying apparatuses may partially overlap. For instance, a single gas cooling apparatus (or set of gas cooling apparatuses) and a single gas humidifying apparatus (or set of gas humidifying apparatuses) may be common to both the first and second sets of gas cooling/heating/humidifying apparatuses. In some embodiments, a single controller is operatively connected to each of the gas cooling/heating/humidifying apparatuses in the gas preparation device and is configured to selectively and separately control the degree to which the gas cooling apparatus(es) cool a gas (or mixture of gases), the degree to which the gas heating apparatus(es) heat a gas (or mixture of gases) and the degree to which the gas humidifying apparatus(es) humidify a gas (or mixture of gases). In some embodiments, a single controller is operatively connected to each of the gas cooling/heating/humidifying apparatuses in the gas preparation device and is configured to selectively and separately control the degree to which each gas cooling/heating/humidifying apparatus cools/heats/humidifies a gas (or mixture of gases).

Controllers of the present invention may be configured to activate the gas cooling apparatus(es), gas heating apparatus(es) and/or gas humidifying apparatus(es) to generate gas flows having any suitable thermal characteristics. In some embodiments, the controller(s) is/are configured to generate gas flows having variable thermal characteristics (e.g., variable temperature, variable rate of temperature change, variable thermal conductivity, etc.). For example, in some embodiments, the controller(s) is/are configured to modulate the activity of the gas cooling apparatus(es), gas heating apparatus(es) and/or gas humidifying apparatus(es) in such a way that the temperature profile of gas(es) that cooled, heated and/or humidified by the gas preparation device varies over time and has the same time/temperature parameters as one or more of the thermal waveforms described in International Patent Application Nos. PCT/US2011/065321; PCT/US2011/065328; PCT/US2011/065338; PCT/US2011/065396 and U.S. patent application Ser. Nos. 13/990,274; 13/990,622; 13/990,912; 13/994,266. In other words, the controller(s) may be configured to regulate the activity of the gas cooling apparatus(es), gas heating apparatus(es) and/or gas humidifying apparatus(es) so as to generate gas flows that constitute time-varying thermal waveforms. In some embodiments, the controller(s) is/are configured to control a first gas cooling apparatus (or set of gas cooling apparatuses) and/or a first gas heating apparatus (or set of apparatuses) to produce a first actively controlled, time-varying thermal waveform (or set of actively controlled, time-varying thermal waveforms) and to control a second gas cooling apparatus (or set of gas cooling apparatuses) and/or a second gas heating apparatus (or set of apparatuses) to produce a second actively controlled, time-varying thermal waveform (or set of actively controlled, time-varying thermal waveforms).

Controllers of the present invention may be configured to activate the gas cooling apparatus(es), gas heating apparatus(es) and/or gas humidifying apparatus(es) in accordance with a prescription (e.g., a prescription comprising instructions for delivering one or more time-varying thermal waveforms to the external auditory canal(s) of a subject). For example, the controller(s) may be configured to control a first gas cooling apparatus (or set of gas cooling apparatuses) and/or a first gas heating apparatus (or set of apparatuses) to produce a first prescribed waveform (or set of waveforms) for delivery to the left external auditory canal of a subject and to control a second gas cooling apparatus (or set of gas cooling apparatuses) and/or a second gas heating apparatus (or set of apparatuses) to produce a second prescribed waveform (or set of waveforms) for delivery to the right external auditory canal of a subject.

Controllers of the present invention may be operatively connected to the gas cooling apparatus(es), gas heating apparatus(es) and/or gas humidifying apparatus(es) via any suitable connection(s). In some embodiments, the controller(s) is/are connected to the gas cooling apparatus(es), gas heating apparatus(es) and/or gas humidifying apparatus(es) via one or more physical leads (as shown in FIGS. 1 and 4-7). For example, in some embodiments, the controller(s) is/are connected to the gas cooling apparatus(es) via one or more gas cooling leads, to the gas heating apparatus(es) via one or more gas heating leads and/or to the gas humidifying apparatus(es) via one or more gas humidifying leads. In some embodiments, the controller(s) is/are wirelessly connected to the gas cooling apparatus(es), gas heating apparatus(es) and/or gas humidifying apparatus(es) (as shown in FIGS. 2-3). For example, in some embodiments, the controller(s) is/are connected to gas cooling apparatus(es), gas heating apparatus(es) and/or gas humidifying apparatus(es) using a radiofrequency transceiver or a Bluetooth connection.

Controllers of the present invention may be configured to activate the gas cooling apparatus(es), gas heating apparatus(es) and/or gas humidifying apparatus(es) in any suitable manner, including, but not limited to, activation with direct current and/or electrical pulses. In some embodiments, the controller(s) is/are configured to control the gas cooling apparatus(es), gas heating apparatus(es) and/or gas humidifying apparatus(es) in a continuous or substantially continuous manner, adjusting one or more parameters of activation (e.g., magnitude, duration, etc.) to produce gas flows having the desired characteristic(s) (e.g., thermal and/or humidity characteristics). For example, each controller may be configured to continuously or substantially continuously activates each of the gas cooling apparatus(es), gas heating apparatus(es) and/or gas humidifying apparatus(es) with which it is operatively connected and to generate gas flows having different thermal and/or humidity characteristics by modulating the level of activation applied to each gas cooling apparatus, gas heating apparatus and/or gas humidifying apparatus. In some embodiments, the gas cooling apparatus(es) are activated at a constant (or substantially constant) rate such that gas(es) flowing from the gas cooling apparatus(es) has/have a uniform (or substantially uniform) temperature profile (e.g., is always about 10° C.), and the controller(s) is/are configured to generate time-varying thermal waveforms by modulating the level of activation applied to the gas heating apparatus(es).

Controllers of the present invention may be configured to control the flow rate(s) of gas(es) as they move through the gas preparation device. In some embodiments, the controller(s) is/are configured to control the flow rate of a gas (or mixture of gases) such that it has a flow rate in the range of about 1 to about 20 liters per minute or more (e.g., about 1, 2, 3, 4, 5, 6, 7, 8, 9, 10, 11, 12, 13, 14, 15, 16, 17, 18, 19, 20 liters per minute or more). In some embodiments, the controller(s) is/are configured to control the flow rate of a gas (or mixture of gases) such that it has a flow rate in the range of about 5 to about 10 liters per minute or more (e.g., about 5, 6, 7, 8, 9, 10 liters per minute or more). In some embodiments, the controller(s) is/are configured to control the flow rate(s) of a gas (or mixtures of gases) by regulating the rate(s) at which the gas(es) enter the gas preparation device and/or by regulating the rate(s) at which the gas(es) exit the gas preparation device. For example, in some embodiments, the controller(s) is/are configured to control the flow rate(s) of a gas (or mixture of gases) by controlling the flow rate of the gas (or mixture of gases) as it is transferred from a gas supply line to a gas intake (by decreasing the pressure of the gas(es) within the gas supply line and/or restricting the size of the air intake, for example). Likewise, in some embodiments, the controller(s) is/are configured to control the flow rate(s) of a gas (or mixture of gases) by controlling the flow rate of the gas (or mixture of gases) as it is transferred from a gas outlet to a conduit (by restricting the size of the gas outlet and/or conduit, for example).

Controllers of the present invention may be configured to receive and/or transmit any suitable data, including, but not limited to, data associated with the parameters, indications and/or approvals of one or more thermal waveforms; data associated with one or more prescriptions; sensor feedback data; data associated with the delivery of one or more thermal waveforms; physician feedback data; and/or subject information.

Controllers of the present invention may comprise any suitable database(s), including, but not limited to, databases configured to receive, store and/or transmit data associated with the parameters, indications and/or approvals of one or more thermal waveforms; data associated with one or more prescriptions; feedback data; data associated with the delivery of one or more thermal waveforms; physician feedback data; and/or subject information. In some embodiments, the controller(s) comprise(s) a waveform database configured to receive, store and/or transmit data associated with the parameters, indications and/or approvals of one or more thermal waveforms (e.g, one or more idealized thermal waveforms); a prescription database configured to receive, transmit and/or store one or more prescriptions; a feedback database configured to receive, transmit and/or store feedback data; and/or a patient history database configured to receive, transmit and/or store patient information. For example, in some embodiments, the controller comprises a waveform database, a prescription database, a feedback database and/or a patient history database as described in International Patent Application No. PCT/US2011/065456 and U.S. patent application Ser. No. 13/994,276.

Controllers of the present invention may comprise any suitable application, including, but not limited to, applications configured to regulate the activity of the cooling/heating/humidifying apparatus(es); to retrieve and/or transmit data; to display information; to accept user input; to analyze data; to generate alert messages; to generate audible tones; to generate visual alerts; to prevent unauthorized use of the gas preparation device and/or to deactivate the gas preparation device. In some embodiments, each controller comprises a control module configured to regulate the activity of one or more cooling apparatuses, one or more heating apparatuses and/or one or more humidifying apparatuses within the gas preparation device; a network module configured to receive, retrieve and/or transmit data; a GUI module configured to display information and/or to accept user input; a feedback module configured to receive, transmit and/or analyze data; an alert generation module configured to generate one or more alert messages; a tone generation module configured to produce audible tones; a visual indicator module configured to notify a user of the existence of an unread/unviewed alert message and/or to notify the user that a treatment session is in progress; a security module configured to prevent unauthorized use of the controller (i.e., to prevent unauthorized persons from using the gas preparation device, to prevent authorized persons from using the gas preparation device in an unauthorized manner, etc.); and/or a safety module configured to deactivate the gas preparation device (or one or more components thereof) in the event of a system malfunction and/or failure. For example, in some embodiments, one or more of the controllers comprises a control module, a network module, a GUI module, a feedback module, an alert generation module, a tone generation module, a visual indicator module, a security module, and/or a safety module as described in International Patent Application No. PCT/US2011/065456 and U.S. patent application Ser. No. 13/994,276.

Controllers of the present invention may receive and/or transmit data over any suitable wired or wireless communications channel, including, but not limited to, a LAN, the Internet, a public telephone switching network, Bluetooth, WLAN and the like.

Controllers of the present invention may receive and/or transmit data from/to any suitable device, including, but not limited to, temperature sensors, humidity sensors, oxygen sensors, gas pressure sensors, gas velocity sensors, devices configured to generate and/or modify instructions for delivering one or more thermal waveforms to the vestibular system and/or the nervous system of a subject, and portable memory devices (e.g., an SD memory card). In some embodiments, the controller(s) is/are configured to receive sensor feedback data from one or more sensors (e.g., one or more temperature sensors and/or one or more humidity sensors) positioned in/on the gas preparation device of which the controller(s) is/are a part and/or one or more sensors (e.g., one or more temperature sensors and/or one or more humidity sensors) positioned in/on a conduit that is operatively connected to the gas preparation device of which the controller(s) is/are a part. In some embodiments, the controller(s) is/are configured to receive data associated with one or more prescriptions from a physician control device (as described in International Patent Application No. PCT/US2011/065456 and U.S. patent application Ser. No. 13/994,276). In some embodiments, the controller(s) is/are configured to receive data associated with the parameters, indications and/or approvals of one or more thermal waveforms (e.g., one or more idealized thermal waveforms) from a physician control device, a registry and/or a portable memory device (e.g., an SD memory card); to receive one or more prescriptions from a physician control device, a registry and/or a portable memory device (e.g., an SD memory card); to receive sensor feedback data from one or more sensors; to receive data associated with the delivery of one or more thermal waveforms (e.g., one or more idealized thermal waveforms) from one or more sensors; to transmit data associated with the delivery of one or more thermal waveforms (e.g., idealized thermal waveforms) to a physician control device, a registry and/or a portable memory device (e.g., an SD memory card); to transmit subject feedback data to a physician control device, a registry and/or a portable memory device (e.g., an SD memory card) and/or to transmit subject information to a physician control device, a registry and/or a portable memory device (e.g., an SD memory card).

Controllers of the present invention may comprise any suitable memory. In some embodiments, the controller comprises one or more of the following types of memory devices: cache, ROM, PROM, EPROM, EEPROM, flash memory, SRAM and DRAM.

Controllers of the present invention may comprise any suitable processor. As will be appreciated by one of skill in the art, the processor may be any commercially available or custom microprocessor.

Controllers of the present invention may comprise any suitable power supply. In some embodiments, the power supply comprises an internal power supply (e.g., one or more rechargeable batteries that may be recharged without first being removed from the controller).

Controllers of the present invention may comprise any suitable software and/or data, including, but not limited to, an operating system, applications, data and input/output (I/O) drivers.

Controllers of the present invention may utilize any suitable operating system, including, but not limited to, OS/2, AIX, OS/390 or System390 from International Business Machines Corp. (Armonk, N.Y.), Window CE, Windows NT, Windows95, Windows98, Windows2000, Windows 7 or Windows Vista from Microsoft Corp. (Redmond, Wash.), Mac OS from Apple, Inc. (Cupertino, Calif.), Unix, Linux or Android.

Gas preparation devices of the present invention may comprise any suitable sensor(s), including, but not limited to, temperature sensors (e.g., thermistors, thermocouples, resistive temperature devices), humidity sensors, oxygen sensors, gas pressure sensors and gas velocity sensors.

Sensors may be positioned at any suitable location in/on the gas preparation device, including, but not limited to, in/on the gas intake(s), in/on the gas outlet(s) and in/on the conduit(s) connecting a first gas cooling/heating/humidifying apparatus to one or more other gas cooling/heating/humidifying apparatuses. In some embodiments, one or more sensors (e.g., one or more temperature sensors and/or one or more humidity sensors) is/are positioned so as to detect one or more characteristics (e.g., temperature and/or humidity) of a gas (or mixture of gases) as it enters the gas preparation device; before it is cooled, heated or humidified; after it has been cooled, but before it has been heated and/or humidified; after it has been cooled and humidified, but before it has been heated; after it has been cooled and heated, but before it has been humidified; after it has been heated, but before it has been cooled and/or humidified; after it has been heated and humidified, but before it has been cooled; after it has been heated and cooled, but before it has been humidified; after it has been humidified, but before it has been cooled and/or heated; after it has been humidified and heated, but before it has been cooled; after it has been humidified and cooled, but before it has been heated; after it has been cooled, heated and humidified; and/or as it exits the gas preparation device.

Sensors may be operatively connected to the controller(s) via any suitable connection(s). In some embodiments, the sensor(s) is/are connected to the controller(s) via one or more physical leads (as shown in FIGS. 1, 3 and 6). For example, in some embodiments, the controller(s) is/are connected to one or more temperature sensors via one or more temperature sensor leads and/or to one or more humidity sensors via one or more humidity sensor leads. In some embodiments, the sensor(s) is/are wirelessly connected to the controller(s) (as shown in FIGS. 2, 4-5 and 7). For example, in some embodiments, the controller(s) is/are connected to one or more temperature sensors and/or one or more humidity sensors using a radiofrequency transceiver or a Bluetooth connection.

The present invention also provides systems for delivering thermal stimulation to the external auditory canal(s) of a subject. In some embodiments, the system comprises, consists essentially of or consists of an gas preparation device configured to cool, heat and/or humidify a gas (or mixture of gases) and one or more conduits configured to deliver the cooled, heated and/or humidified gas(es) to the external auditory canal(s) of a subject. In some embodiments, the system comprises one or more sensors (e.g., one or more temperature sensors and/or one or more humidity sensors) configured to detect one or more characteristics (e.g., temperature and/or humidity) of the cooled, heated and/or humidified gas(es) and to provide feedback to the gas preparation device. The gas preparation device may utilize feedback provided by the temperature sensor(s) to modulate the degree to which the gas(es) is/are cooled, heated and/or humidified.

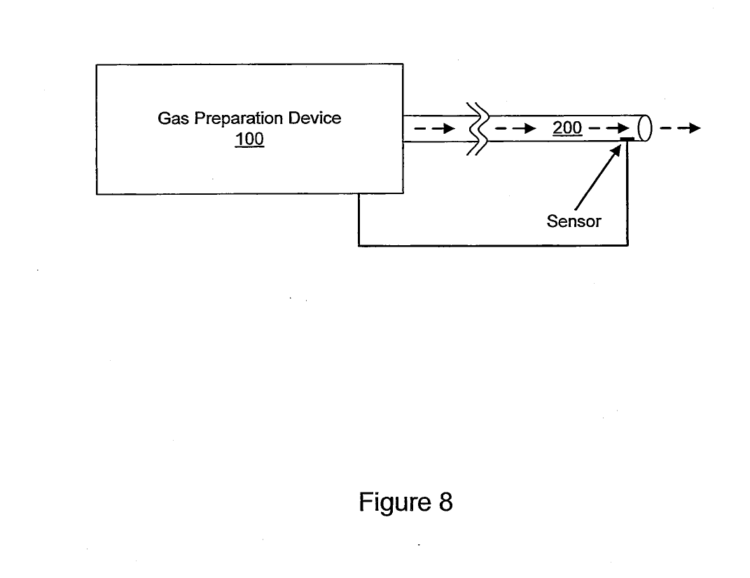

As shown in FIG. 8, in some embodiments, the system comprises, consists essentially of or consists of a gas preparation device 100 and a conduit 200.

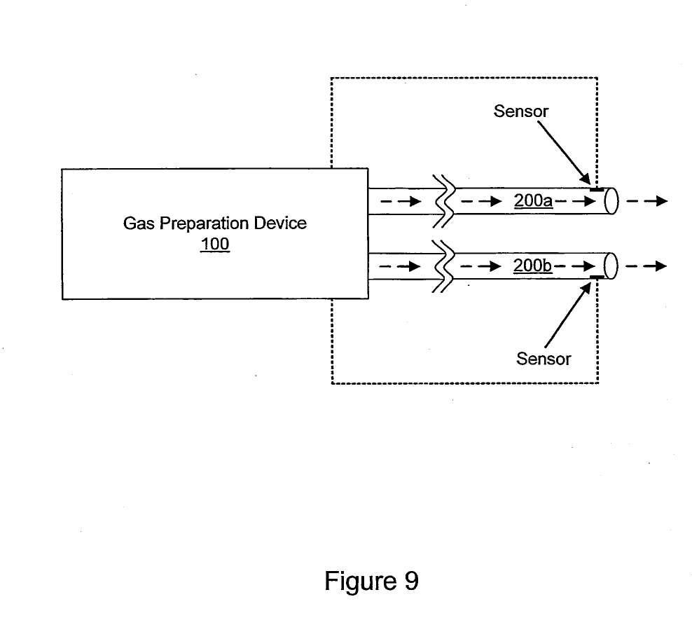

As shown in FIG. 9, in some embodiments, the system comprises, consists essentially of or consists of a gas preparation device 100 and a pair of conduits 200a, 200b. For example, in some embodiments, the system comprises, consists essentially of or consists of a gas preparation device 100, a first conduit 200a configured to deliver cooled, heated and/or humidified gas(es) to the left external auditory canal of a subject, and a second conduit 200b configured to deliver cooled, heated and/or humidified gas(es) to the right external auditory canal of the subject.

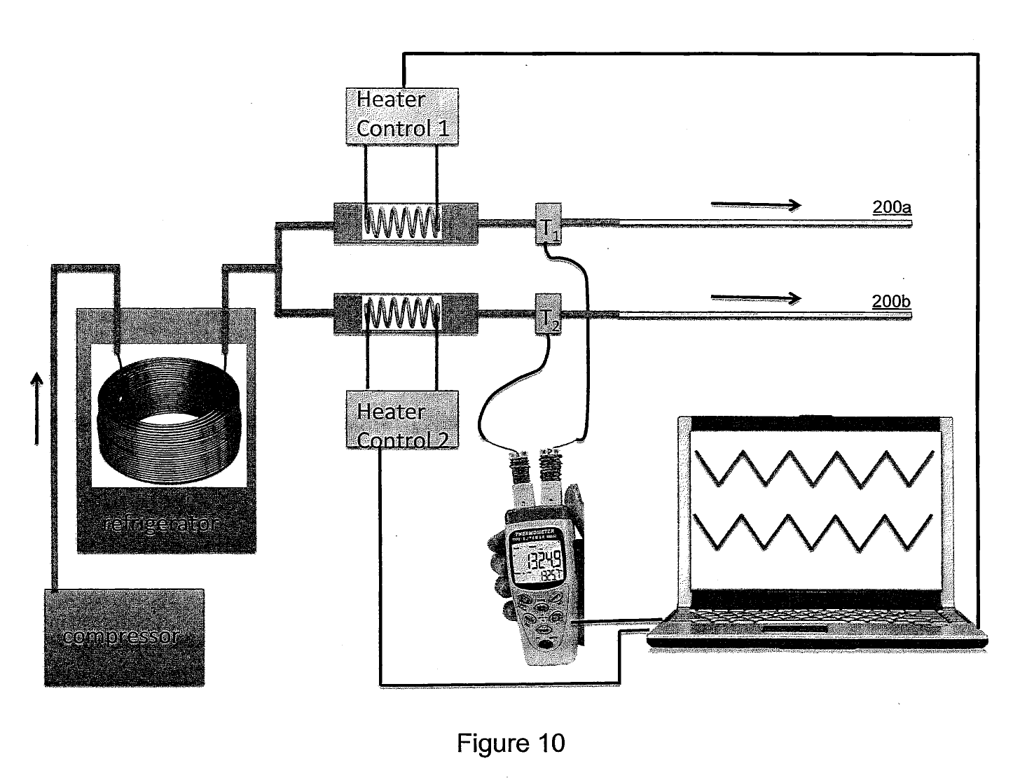

As shown in FIG. 10, in some embodiments, the system comprises, consists essentially of or consists of a pair of conduits 200a, 200b for delivering cooled and heated gas(es) to the external auditory canals of a subject and a gas preparation device comprising a gas compressor, a gas refrigeration element, and a gas heating apparatus that comprises two resistive heating coils, two temperature sensors T₁, T₂ and a controller configured to receive feedback from the temperature sensors T₁, T₂ and to control the resistive heating coils responsive to that feedback so as to deliver a first prescribed thermal waveform via one conduit 200a and a second prescribed thermal waveform via the other conduit 200b.

Systems of present invention may comprise any suitable gas preparation device(s), including, but not limited to, gas preparation devices of the present invention.

Systems of present invention may comprise any suitable conduit(s). In some embodiments, the conduit(s) are pliable tubes. For example, in some embodiments, the system comprises one or more conduits comprising, consisting essentially of or consisting of medical grade plastic tubing. In some embodiments, the conduit(s) is/are MRI-compatible (i.e., “MR-Safe” or “MR-Conditional” as defined by ASTM Standard F2503-13 “Standard Practice for Marking Medical Devices and Other Items for Safety in the Magnetic Resonance Environment”). In some embodiments, at least a portion of the conduit(s) is insulated to reduce heat loss/gain as gas(es) travel(s) through the conduit(s).

Conduits of the present invention may be configured to in any suitable conformation. In some embodiments, the distal end of a conduit (i.e., the end proximate the external auditory canal of the subject) is dimensioned so as to be at least partially insertable into the external auditory canal of the subject. In some embodiments, the distal end of the conduit is dimensioned so as to at least partially seal the external auditory canal of the subject. For example, in some embodiments, the distal end of the conduit comprises a flexible material that is at least partially conformable to the external auditory canal during use. Alternatively, in some embodiments, the distal end of the conduit comprises a rigid (or semi-rigid) material that is so dimensioned as to the snugly fit the external auditory canal during use. It will be understood that such earpieces may be custom-manufactured to fit a subject's external auditory canal (via three-dimensional printing, for example). In some embodiments, the distal end of the conduit comprises a mechanism whereby the conduit may be removably affixed to the ear of the subject. For example, in some embodiments, the distal end of the conduit may comprise a segment configured to fit between the outer ear and head of the subject, thereby securing the distal end of the conduit in the proper location within or adjacent to the external auditory ear canal of the subject. In some embodiments, the conduit comprises one or more venting mechanisms (e.g., holes) that allow gas(es) to escape the external auditory ear canal, thereby reducing pressure buildup within the external auditory canal during use.

The conduit(s) may be operably connected to the gas preparation device using any suitable means. In some embodiments, the conduit(s) is/are inserted into and/or affixed to the gas outlet(s) of the gas preparation device. In preferred embodiments, each connection between the gas preparation device and a conduit comprises an airtight seal.

Systems of the present invention may comprise any suitable sensor(s), including, but not limited to, temperature sensors (e.g., thermistors, thermocouples, resistive temperature devices), humidity sensors, oxygen sensors, gas pressure sensors and gas velocity sensors.

Sensors may be positioned at any suitable location, including, but not limited to, in/on the gas preparation device, in/on the conduits and in the external auditory canal of the subject. In some embodiments, one or more sensors (e.g., one or more temperature sensors and/or one or more humidity sensors) is/are positioned in/on the gas preparation device to detect one or more characteristics (e.g., temperature and/or humidity) of a gas (or mixture of gases) as it exits the gas preparation device. In some embodiments, one or more sensors (e.g., one or more temperature sensors and/or one or more humidity sensors) is/are positioned between the gas preparation device and the conduit(s) to detect one or more characteristics (e.g., temperature and/or humidity) of a gas (or mixture of gases) as it passes from the gas preparation device to the conduit(s). In some embodiments, one or more sensors (e.g., one or more temperature sensors and/or one or more humidity sensors) is/are positioned in/on the conduit(s) to detect one or more characteristics (e.g., temperature and/or humidity) of a gas (or mixture of gases) as is passes through the conduit(s) and/or as exits the conduit(s) and is delivered to the external auditory canal(s) of the subject.

Sensors may be operatively connected to the gas preparation device via any suitable connection(s). In some embodiments, the sensor(s) is/are connected to the gas preparation device (i.e., to the controller(s) within the gas preparation device) via one or more physical leads (as shown in FIG. 8). For example, in some embodiments, a temperature sensor in the conduit is connected to the gas preparation device's controller via a temperature sensor lead. In some embodiments, the sensor(s) is/are wirelessly connected to the gas preparation device (i.e., to the controller(s) within the gas preparation device) (as shown in FIG. 9). For example, in some embodiments, the temperature sensor(s) in each conduit is/are connected to the controller(s) using a radiofrequency transceiver or a Bluetooth connection.

Systems of the present invention may be configured to deliver any suitable gases or mixture of gases, including, but not limited to, oxygen, nitrogen, carbon dioxide, helium, neon, argon, krypton, xenon and combinations thereof. In some embodiments, the system is configured to deliver ambient air that has been cooled, heated and/or humidified.

Systems of the present invention may comprise any suitable gas supply(ies), including, but not limited to, positive pressure gas supplies. In some embodiments, the gas supply comprises a fan, a gas purifier (e.g., a gas filtration device/system configured to remove particulate matter and/or volatile chemicals, to kill gasborne microbes, etc.), a gas humidifier, a gas dehumidifier, a gas compressor, a gas line (e.g., an oxygen supply line in a hospital) and/or a gas storage vessel (e.g., a gas canister).

The gas supply(ies) may be operably connected to the gas preparation device using any suitable means. In some embodiments, the gas supply(ies) is/are inserted into and/or affixed to the gas intake(s) of the gas preparation device. In preferred embodiments, each connection between the gas preparation device and a gas supply comprises an airtight seal.

Systems of the present invention may be configured to deliver a gas (or mixture of gases) of any suitable temperature to the external auditory canal(s) of a subject. In some embodiments, the system is configured to deliver a gas (or mixture of gases) having a temperature in the range of about 10 to about 50 degrees Centigrade (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees Centigrade) when it contacts the external auditory canal(s) of the subject. Because gas(es) cooled and/or heated by the gas preparation device may lose/gain heat as it/they travel(s) through the conduit(s) to the external auditory canal(s) of the subject, in some embodiments, the system is configured to cool the gas(es) below 10 degrees Centigrade and/or to heat the gas(es) above 50 degrees Centigrade. For example, in some embodiments, the system is configured to cool the gas(es) to about 1, 2, 3, 4, 5, 6, 7, 8 or 9 degrees Centigrade and/or to heat the gas(es) to about 51, 52, 53, 54, 55, 56, 57, 59, 59 or 60 degrees Centigrade.

Systems of the present invention may be configured to deliver any suitable thermal stimulus. In some embodiments, the system is configured to deliver one or more thermal waveforms (e.g., one or more actively controlled, time-varying thermal waveforms) to the vestibular system and/or the nervous system of a subject.

Systems of the present invention may be configured to deliver any suitable thermal waveform or combination of thermal waveforms, including, but not limited to, those described in International Patent Application Nos. PCT/US2011/065321; PCT/US2011/065328; PCT/US2011/065338; PCT/US2011/065396 and U.S. patent application Ser. Nos. 13/990,274; 13/990,622; 13/990,912; 13/994,266. In some embodiments, the system is configured to deliver one or more actively controlled, time-varying thermal waveforms to the vestibular system and/or the nervous system of a subject. In some embodiments, the system is configured to generate a prescription comprising a set of instructions for delivering one or more thermal waveforms to the vestibular system and/or the nervous system of a subject and to deliver the prescribed thermal waveform(s) to the vestibular system and/or the nervous system of said subject.

Systems of the present invention may be configured to change the temperature(s) of a subject's external auditory canal(s) to any suitable degree and at any suitable rate. In some embodiments, the system is configured to lower the temperature of a subject's external auditory canal by about 1 to about 30 degrees Centigrade or more (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 degrees Centigrade or more). In some embodiments, the system is configured to lower the temperature of a subject's external auditory canal by about 5 to about 20 degrees Centigrade or more (e.g., about 5, 6, 7, 8, 9, 10, 11, 12, 13, 14, 15, 16, 17, 18, 19 or 20 degrees Centigrade or more). In some embodiments, the system is configured to raise the temperature of a subject's external auditory canal by about 1 to about 10 degrees Centigrade or more (e.g., about 1, 2, 3, 4, 5, 6, 7, 8, 9 or 10 degrees Centigrade or more). In some embodiments, the system is configured to raise the temperature of a subject's external auditory canal by about 5 to about 10 degrees Centigrade or more (e.g., about 5, 6, 7, 8, 9 or 10 degrees Centigrade or more). In some embodiments, the system is configured to change the temperature of a subject's external auditory canal by about 5 to about 35 degrees Centigrade or more per minute (e.g., about 5, 6, 7, 8, 9, 10, 11, 12, 13, 14, 15, 16, 17, 18, 19, 20, 22, 23, 24, 25, 26, 27, 28, 29, 30, 31, 32, 33, 34, 35 or more degrees Centigrade per minute). In some embodiments, the system is configured to change the temperature of a subject's external auditory canal by about 10 to about 30 degrees Centigrade or more per minute (e.g., about 10, 11, 12, 13, 14, 15, 16, 17, 18, 19, 20, 22, 23, 24, 25, 26, 27, 28, 29, 30 or more degrees Centigrade per minute).

Systems of the present invention may be configured to deliver gas(es) at any suitable flow rate(s). In some embodiments, the system is configured to deliver a gas (or mixture of gases) at a flow rate in the range of about 1 to about 20 liters per minute or more (e.g., about 1, 2, 3, 4, 5, 6, 7, 8, 9, 10, 11, 12, 13, 14, 15, 16, 17, 18, 19, 20 liters per minute or more). In some embodiments, the system is configured to deliver a gas (or mixture of gases) at a flow rate in the range of about 5 to about 10 liters per minute or more (e.g., about 5, 6, 7, 8, 9, 10 liters per minute or more).

The present invention also provides methods of delivering thermal stimulation. In some embodiments, the method comprises, consists essentially of or consists of administering one or more cooled, heated and/or humidified gases to the external auditory canal(s) of a subject. In some embodiments, the method comprises cooling, heating and/or humidifying the gas(es) prior to administration.

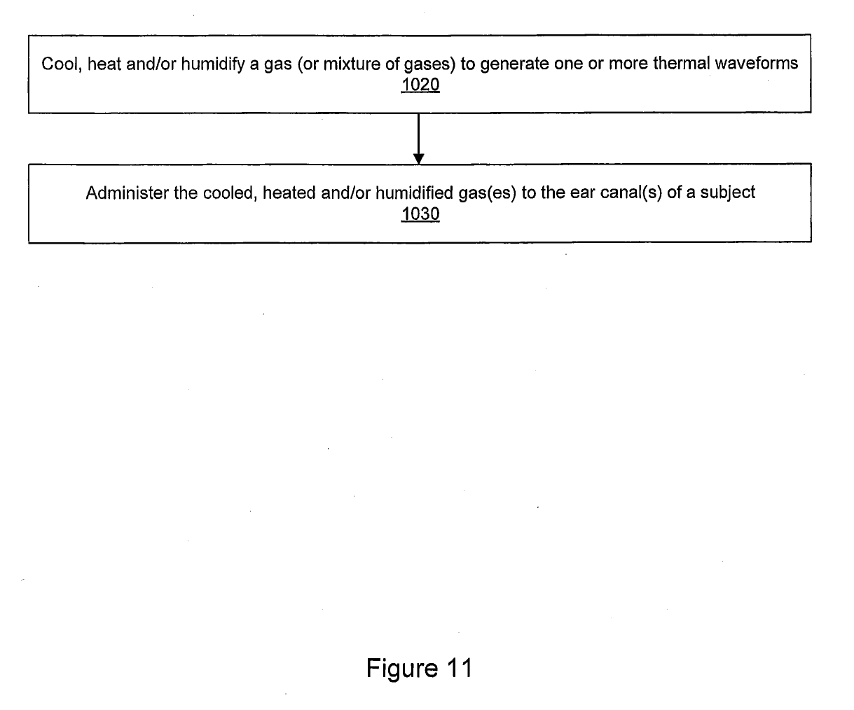

As shown in FIG. 11, in some embodiments, the method comprises, consists essentially of or consists of cooling, heating and/or humidifying a gas (or mixture of gases) to generate a thermal waveform (e.g., an actively controlled, time-varying thermal waveform) 1020; and administering the cooled, heated and/or humidified gas(es) to the external auditory canal(s) of a subject 1030.

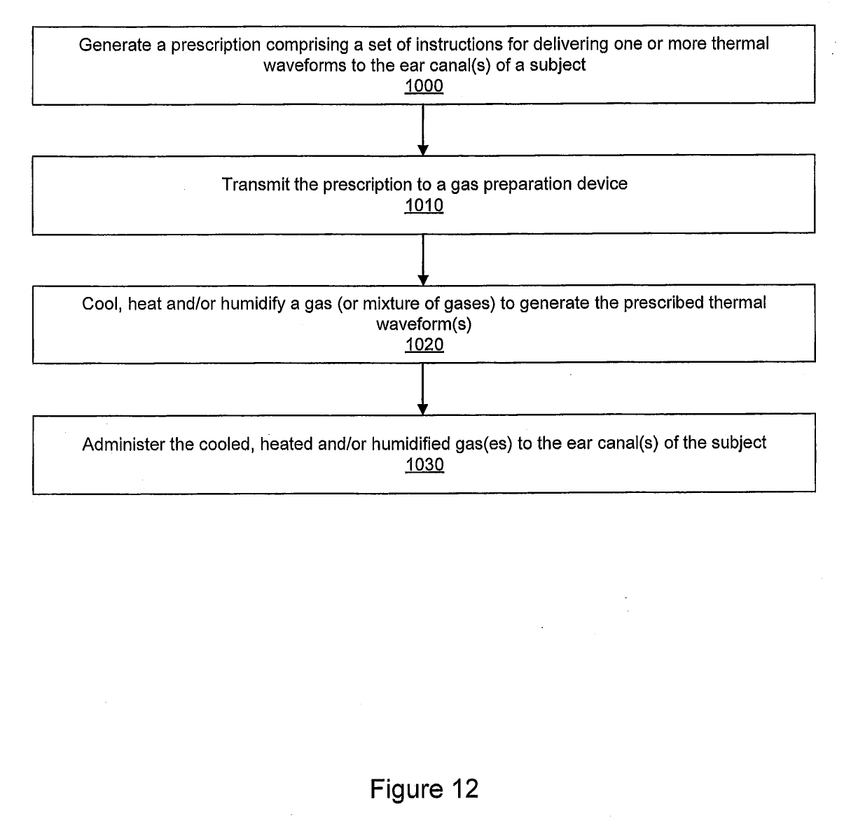

As shown in FIG. 12, in some embodiments, the method comprises, consists essentially of or consists of generating a prescription comprising a set of instructions for delivering one or more thermal waveforms to the external auditory canal(s) of a subject 1000; transmitting the prescription to a gas preparation device 1010; cooling, heating and/or humidifying a gas (or mixture of gases) to generate the prescribed thermal waveform(s) 1020; and administering the cooled, heated and/or humidified gas(es) to the external auditory canal(s) of the subject 1030.

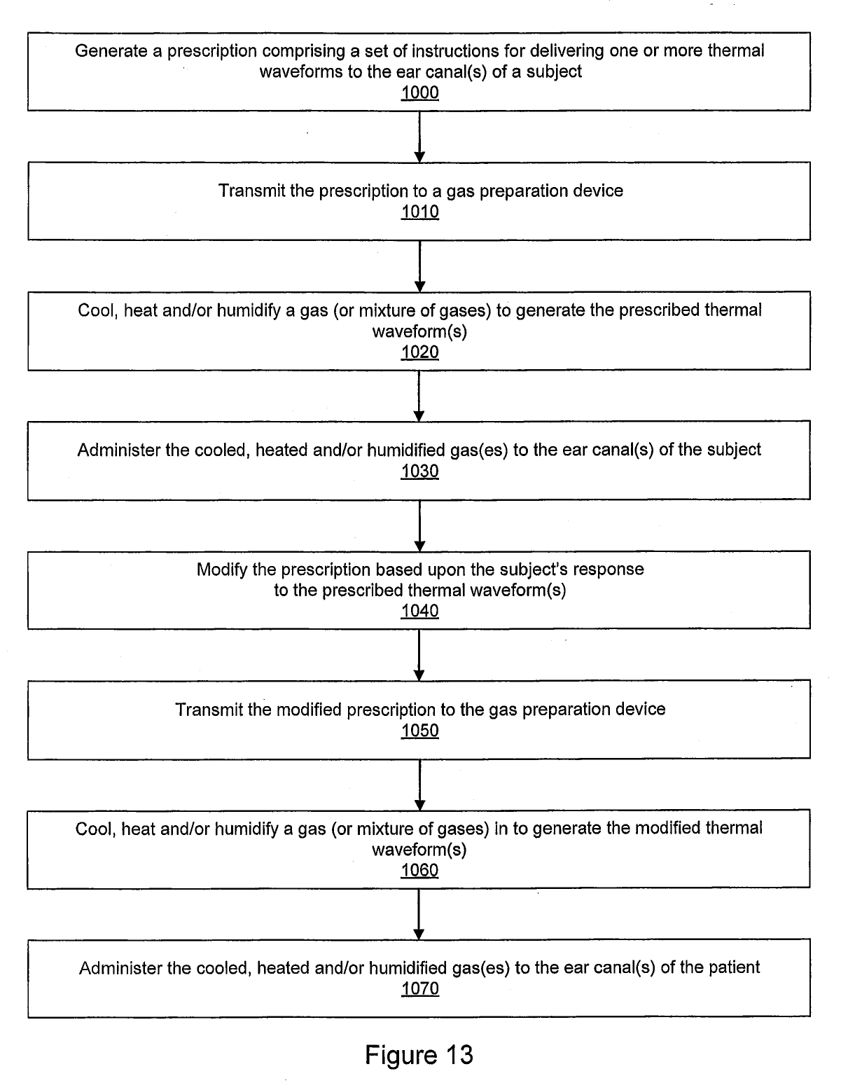

As shown in FIG. 13, in some embodiments, the method comprises, consists essentially of or consists of generating a prescription comprising a set of instructions for delivering one or more thermal waveforms to the external auditory canal(s) of a subject 1000; transmitting the prescription to a gas preparation device 1010; cooling, heating and/or humidifying a gas (or mixture of gases) to generate the prescribed thermal waveform(s) 1020; and administering the cooled, heated and/or humidified gas(es) to the external auditory canal(s) of the subject 1030; modifying the prescription based upon the subject's response to the prescribed thermal waveform(s) 1040; transmitting the modified prescription to the gas preparation device 1050; cooling, heating and/or humidifying a gas (or mixture of gases) to generate the prescribed thermal waveform(s) as modified 1060; and administering the cooled, heated and/or humidified gas(es) to the external auditory canal(s) of the subject 1070.

Any suitable gas(es) or mixture(s) of gases may be administered, including, but not limited to, oxygen, nitrogen, carbon dioxide, helium, neon, argon, krypton, xenon and combinations thereof. In some embodiments, administering one or more cooled, heated and/or humidified gases to the external auditory canal of a subject comprises administering ambient air that has been cooled, heated and/or humidified.

Gases (or mixtures of gases) may be administered using any suitable device/system, including, but not limited to, devices and systems of the present invention. In some embodiments, administering one or more cooled, heated and/or humidified gases to the external auditory canal of a subject comprises placing a conduit of the present invention adjacent to or in the external auditory ear canal of the subject and passing one or more cooled, heated and/or humidified gases through the conduit.

Gases (or mixtures of gases) of any suitable temperature(s) may be administered. In some embodiments, the gas (or mixture of gases) administered has a temperature in the range of about 1 to about 60 degrees Centigrade (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 degrees Centigrade). In some embodiments, the gas (or mixtures of gases) administered has a temperature in the range of about 5 to about 55 degrees Centigrade (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 degrees Centigrade). In some embodiments, the gas (or mixture of gases) administered has a temperature in the range of about 10 to about 50 degrees Centigrade (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees Centigrade).

Gases (or mixtures of gases) of any suitable humidity may be administered. In some embodiments, the gas (or mixture of gases) administered has a humidity in the range about 5 to about 95 percent relative/specific humidity (e.g., about 5, 6, 7, 8, 9, 10, 11, 12, 13, 14, 15, 16, 17, 18, 19, 20, 25, 30, 35, 40, 45, 50, 55, 60, 65, 70, 75, 76, 77, 78, 79, 80, 81, 82, 83, 84, 85, 86, 87, 88, 89, 90, 91, 92, 93, 94 or 95 percent relative/specific humidity). In some embodiments, the gas (or mixture of gases) administered has a humidity in the range about 10 to about 90 percent relative/specific humidity (e.g., about 10, 11, 12, 13, 14, 15, 16, 17, 18, 19, 20, 25, 30, 35, 40, 45, 50, 55, 60, 65, 70, 75, 76, 77, 78, 79, 80, 81, 82, 83, 84, 85, 86, 87, 88, 89 or 90 percent relative/specific humidity).

Gases (or mixtures of gases) may be cooled, heated and/or humidified using any suitable protocol. In some embodiments, a gas (or mixtures of gases) is cooled, then humidified, then heated. In some embodiments, a gas (or mixtures of gases) is cooled, then heated, then humidified.

Gases (or mixtures of gases) may be cooled, heated and/or humidified using any suitable device/system, including, but not limited to, devices and systems of the present invention.

Gases (or mixtures of gases) may be cooled to any suitable temperature(s). In some embodiments, the gas (or mixtures of gases) is cooled to a temperature in the range of about 1 to about 60 degrees Centigrade (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 degrees Centigrade). In some embodiments, the gas (or mixtures of gases) is cooled to a temperature in the range of about 5 to about 55 degrees Centigrade (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 degrees Centigrade). In some embodiments, the gas (or mixtures of gases) is cooled to a temperature in the range of about 10 to about 50 degrees Centigrade (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees Centigrade).

Gases (or mixtures of gases) may be heated to any suitable temperature(s). In some embodiments, the gas (or mixtures of gases) is heated to a temperature in the range of about 1 to about 60 degrees Centigrade (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 degrees Centigrade). In some embodiments, the gas (or mixtures of gases) is heated to a temperature in the range of about 5 to about 55 degrees Centigrade (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 degrees Centigrade). In some embodiments, the gas (or mixtures of gases) is heated to a temperature in the range of about 10 to about 50 degrees Centigrade (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees Centigrade).

Gases (or mixtures of gases) may be humidified to any suitable humidity. In some embodiments, the gas (or mixtures of gases) is humidified to a humidity in the range about 5 to about 95 percent relative/specific humidity (e.g., about 5, 6, 7, 8, 9, 10, 11, 12, 13, 14, 15, 16, 17, 18, 19, 20, 25, 30, 35, 40, 45, 50, 55, 60, 65, 70, 75, 76, 77, 78, 79, 80, 81, 82, 83, 84, 85, 86, 87, 88, 89, 90, 91, 92, 93, 94 or 95 percent relative/specific humidity). In some embodiments, the gas (or mixtures of gases) is humidified to a humidity in the range about 10 to about 90 percent relative/specific humidity (e.g., about 10, 11, 12, 13, 14, 15, 16, 17, 18, 19, 20, 25, 30, 35, 40, 45, 50, 55, 60, 65, 70, 75, 76, 77, 78, 79, 80, 81, 82, 83, 84, 85, 86, 87, 88, 89 or 90 percent relative/specific humidity).

Gases (or mixtures of gases) may be cooled, heated and/or humidified to generate any suitable stimulus. In some embodiments, a gas (or mixtures of gases) is cooled, heated and/or humidified so as to generate one or more time-varying thermal waveforms.

Any suitable thermal waveform or combination of thermal waveforms may be generated and delivered, including, but not limited to, those described in International Patent Application Nos. PCT/US2011/065321; PCT/US2011/065328; PCT/US2011/065338; PCT/US2011/065396 and U.S. patent application Ser. Nos. 13/990,274; 13/990,622; 13/990,912; 13/994,266. In some embodiments, one or more actively controlled, time-varying thermal waveforms is generated and delivered to the vestibular system and/or the nervous system of the subject.

Devices, systems and methods of the present invention may be used for any suitable reason, including, but not limited to, the diagnosis, prevention and/or treatment of one or more disorders, and modulation of the autonomic nervous system (e.g., sympathetic-parasympathetic balance, heart rate variability, respiratory rate). Devices, systems and methods of the present invention may be particularly useful for testing/treatment methods involving the use of magnetic resonance imaging because cooled, heated and/or humidified gases (or mixtures of gases) (e.g., cooled, heated and/or humidified air) may be used to stimulate a subject's vestibular and/or nervous systems without adversely affecting the quality of the resultant images.

Devices, systems and methods of the present invention may be used to diagnose, prevent and/or treat any suitable disorder, including, but not limited to, tinnitus, neuropathic pain (e.g., migraine headaches), brain injury (acute brain injury, excitotoxic brain injury, traumatic brain injury, etc.), spinal cord injury, body image or integrity disorders (e.g., spatial neglect), visual intrusive imagery, neuropsychiatric disorders (e.g., depression), bipolar disorder, neurodegenerative disorders (e.g., Parkinson's disease), epilepsy, asthma, dementia, insomnia, stroke, cellular ischemia, metabolic disorders, (e.g., diabetes), anxiety, post-traumatic stress disorder (“PTSD”), addictive disorders, sensory disorders, motor disorders, and cognitive disorders. In some embodiments, devices, systems and methods of the present invention are used to diagnose, prevent and treat one or more symptoms of patients in a minimally conscious and/or persistent vegetative state.

Headaches that may be prevented and/or treated by the devices, systems and methods of the present invention include, but are not limited to, primary headaches (e.g., migraine headaches, tension-type headaches, trigeminal autonomic cephalagias and other primary headaches, such as cough headaches and exertional headaches) and secondary headaches. See, e.g., International Headache Society Classification ICHD-II.

Migraine headaches that may be prevented and/or treated by the devices, systems and methods of the present invention may be acute/episodic/chronic and unilateral/bilateral. The migraine headache may be of any type, including, but not limited to, migraine with aura, migraine without aura, hemiplegic migraine, opthalmoplegic migraine, retinal migraine, basilar artery migraine, abdominal migraine, vestibular migraine and probable migraine. As used herein, the term “vesibular migraine” refers to migraine with associated vestibular symptoms, including, but not limited to, head motion intolerance, unsteadiness, dizziness and vertigo. Vestibular migraine includes, but is not limited to, those conditions sometimes referred to as vertigo with migraine, migraine-associated dizziness, migraine-related vestibulopathy, migrainous vertigo and migraine-related vertigo. See, e.g., Teggi et al., HEADACHE 49:435-444 (2009).

Tension-type headaches that may be prevented and/or treated by the devices, systems and methods of the present invention, include, but are not limited to, infrequent episodic tension-type headaches, frequent episodic tension-type headaches, chronic tension-type headache and probable tension-type headache.

Trigeminal autonomic cephalagias that may be prevented and/or treated by the devices, systems and methods of the present invention, include, but are not limited to, cluster headaches, paroxysmal hemicranias, short-lasting unilateral neuralgiform headache attacks with conjunctival injection and tearing and probable trigeminal autonomic cephalagias. Cluster headache, sometimes referred to as “suicide headache,” is considered different from migraine headache. Cluster headache is a neurological disease that involves, as its most prominent feature, an immense degree of pain. “Cluster” refers to the tendency of these headaches to occur periodically, with active periods interrupted by spontaneous remissions. The cause of the disease is currently unknown. Cluster headaches affect approximately 0.1% of the population, and men are more commonly affected than women (in contrast to migraine headache, where women are more commonly affected than men).

Other primary headaches that may be prevented and/or treated by the devices, systems and methods of the present invention, include, but are not limited to, primary cough headache, primary exertional headache, primary headache associated with sexual activity, hypnic headache, primary thunderclap headache, hemicranias continua and new daily-persistent headache. Sensory disorders that may be treated by the methods and apparatuses of the present invention include, but are not limited to, vertigo, dizziness, seasickness, travel sickness cybersickness, sensory processing disorder, hyperacusis, fibromyalgia, neuropathic pain (including, but not limited to, complex regional pain syndrome, phantom limb pain, thalamic pain syndrome, craniofacial pain, cranial neuropathy, autonomic neuropathy, and peripheral neuropathy (including, but not limited to, entrapment-, heredity-, acute inflammatory-, diabetes-, alcoholism-, industrial toxin-, Leprosy-, Epstein Barr Virus-, liver disease-, ischemia-, and drug-induced neuropathy)), numbness, hemianesthesia, and nerve/root plexus disorders (including, but not limited to, traumatic radiculopathies, neoplastic radiculopathies, vaculitis, and radiation plexopathy).

Motor disorders that may be prevented and/or treated by the devices, systems and methods of the present invention include, but are not limited to, upper motor neuron disorders such as spastic paraplegia, lower motor neuron disorders such as spinal muscular atrophy and bulbar palsy, combined upper and lower motor neuron syndromes such as familial amyotrophic lateral sclerosis and primary lateral sclerosis, aphasia (e.g., post-stroke aphasia), and movement disorders (including, but not limited to, Parkinson's disease, tremor, dystonia, Tourette Syndrome, myoclonus, chorea, nystagmus, spasticity, agraphia, dysgraphia, alien limb syndrome, and drug-induced movement disorders).

Cognitive disorders that may be prevented and/or treated by the devices, systems and methods of the present invention include, but are not limited to, schizophrenia, addiction, anxiety disorders, depression, bipolar disorder, dementia, insomnia, narcolepsy, autism, Alzheimer's disease, anomia, aphasia (e.g., post-stroke aphasia), dysphasia, parosmia, spatial neglect, attention deficit hyperactivity disorder, obsessive compulsive disorder, eating disorders, body image disorders, body integrity disorders, post-traumatic stress disorder, intrusive imagery disorders, and mutism.

Metabolic disorders that may be prevented and/or treated by the devices, systems and methods of the present invention include type I diabetes, type II diabetes, gestational diabetes, hypertension, obesity, etc.

Addiction, addictive disorders, and/or addictive behaviors that may be prevented and/or treated by the devices, systems and methods of the present invention include, but are not limited to, alcohol addiction, tobacco or nicotine addiction (e.g., using the present invention as a smoking cessation aid), drug addictions (e.g., opiates, oxycontin, amphetamines, etc.), food addictions (compulsive eating disorders), etc.

In some embodiments, the subject has two or more of the above disorders/conditions, and both conditions are treated concurrently with the devices, systems and methods of the present invention. For example, a subject with both depression and anxiety (e.g., PTSD) may be diagnosed as having and treated for both disorders, concurrently, with the devices, systems and methods of the present invention.

Although systems and methods of the present invention have heretofore been described with respect to the delivery of thermal stimulation to the external auditory canal(s) of a subject, it is to be understood that systems and methods of the present invention may be used to stimulate any suitable target. Thus, in some embodiments, systems of the present invention are configured to stimulate other bodily targets, such as the nasal cavity of a subject.

The foregoing is illustrative of the present invention, and is not to be construed as limiting thereof. The invention is defined by the following claims, with equivalents of the claims to be included herein.

That which is claimed:

1. A system for delivering thermal stimulation, comprising:
a gas preparation device configured to cool, heat and/or humidify a gas or mixture of gases; and
a conduit configured to deliver the cooled, heated and/or humidified gas or mixture of gases to an external auditory canal of a subject.

2. The system of claim 1, wherein the conduit is dimensioned so as to be at least partially insertable into the external auditory canal of the subject.

3. The system of claim 1, wherein the gas preparation device comprises:
an gas cooling apparatus configured to cool the gas or mixture of gases; and
a controller configured to control the degree to which the gas cooling apparatus cools the gas or mixture of gases.

4. The system of claim 1, wherein the gas preparation device comprises:
an gas heating apparatus configured to heat the gas or mixture of gases; and
a controller configured to control the degree to which the gas heating apparatus heats the gas or mixture of gases.

5. The system of claim 1, wherein the gas preparation device comprises:
an gas humidifier configured to humidify the gas or mixture of gases; and
a controller configured to control the degree to which the gas humidifier humidifies the gas or mixture of gases.

6. The system of claim 1, wherein the gas preparation device comprises:
an gas cooling apparatus configured to cool the gas or mixture of gases;
an gas heating apparatus configured to heat the gas or mixture of gases; and
a controller,
wherein the controller is configured to control the degree to which the gas cooling apparatus cools the gas or mixture of gases and the degree to which the gas heating apparatus heats the gas or mixture of gases.

7. The system of claim 1, wherein the gas preparation device comprises:
an gas cooling apparatus configured to cool the gas or mixture of gases;
an gas humidifier configured to humidify the gas or mixture of gases; and
a controller,
wherein the controller is configured to control the degree to which the gas cooling apparatus cools the gas or mixture of gases and the degree to which the gas humidifier humidifies the gas or mixture of gases.

8. The system of claim 1, wherein the gas preparation device comprises:
an gas heating apparatus configured to heat the gas or mixture of gases;
an gas humidifier configured to humidify the gas or mixture of gases; and
a controller,
wherein the controller is configured to control the degree to which the gas heating apparatus heats the gas or mixture of gases and the degree to which the gas humidifier humidifies the gas or mixture of gases.

9. The system of claim 1, wherein the gas preparation device comprises:
an gas cooling apparatus configured to cool the gas or mixture of gases;
an gas heating apparatus configured to heat the gas or mixture of gases;
an gas humidifier configured to humidify the gas or mixture of gases; and
a controller,
wherein the controller is configured to control the degree to which the gas cooling apparatus cools the gas or mixture of gases, the degree to which the gas heating apparatus heats the gas or mixture of gases, and the degree to which the gas humidifier humidifies the gas or mixture of gases.

10. The system of claim 3, wherein the gas cooling apparatus comprises one or more refrigeration coils.

11. The system of claim 4, wherein the gas heating apparatus comprises one or more resistive heating elements.

12. The system of claim 3, wherein the controller is configured to control the degree to which the gas cooling apparatus cools the gas or mixture of gases so as to deliver one or more thermal waveforms to the external auditory canal of the subject.

13. The system of claim 3, wherein the controller is configured to control the degree to which the gas heating apparatus heats the gas or mixture of gases so as to deliver one or more thermal waveforms to the external auditory canal of the subject.

14. The system of claim 6, wherein the controller is configured to control the degree to which the gas cooling apparatus cools the gas or mixture of gases and the degree to which the gas heating apparatus heats the gas or mixture of gases so as to deliver one or more thermal waveforms to the external auditory canal of the subject.

15. The system of claim 12, wherein the one or more thermal waveforms comprises one or more actively controlled, time-varying thermal waveforms.

16. The system of claim 1, further comprising a temperature sensor configured to detect the temperature of the cooled, heated and/or humidified gas or mixture of gases.

17. The system of claim 1, wherein the gas preparation device is operably connected to a positive pressure gas supply.

18. A method of delivering thermal stimulation, comprising:
administering one or more cooled, heated and/or humidified gases to one or more external auditory canals of a subject.

19. The method of claim 18, further comprising cooling one or more gases to about 5° C.

20. The method of claim 19, further comprising heating the one or more cooled gases to one or more temperatures in a range of about 10° C. to about 55° C.

21. The method of claim 20, further comprising heating one or more gases to about 55° C.

22. The method of claim 21, further comprising cooling the one or more heated gases to one or more temperatures in a range of about 10° C. to about 50° C.

23. The method of claim 18, further comprising humidifying the one or more cooled and/or heated gases to about 10% to about 90% humidity.

24. The method of claim 18, wherein the one or more gases is/are cooled, heated and/or humidified according to a prescription comprising a set of instructions for delivering a thermal stimulus to each of the one or more external auditory canal(s) of the subject.

25. The method of claim 24, wherein each thermal stimulus comprises one or more time-varying thermal waveforms.

26. The method of claim 24, wherein the prescription comprises instructions for delivering a first thermal stimulus to a first external auditory canal of the subject and instructions for delivering a second thermal stimulus different from the first thermal stimulus to a second external auditory canal of the subject.

27. The method of claim 24, further comprising generating the prescription.
